SEQUENCE LISTING (5 sequences)

SEQ ID NO: 1 - DNA, 1530 bp, Homo sapiens
atggtagatggagtgatgattcttcctgtgcttatcatgattgctctcccctcccctagtatggaagatgagaagcccaaggtcaaccccaaactctacatgtgtgtgtgtgaaggtctctcctgcggtaatgaggaccactgtgaaggccagcagtgcttttcctcactgagcatcaacgatggcttccacgtctaccagaaaggctgcttccaggtttatgagcagggaaagatgacctgtaagaccccgccgtcccctggccaagccgtggagtgctgccaaggggactggtgtaacaggaacatcacggcccagctgcccactaaaggaaaatccttccctggaacacagaatttccacttggaggttggcctcattattctctctgtagtgttcgcagtatgtcttttagcctgcctgctgggagttgctctccgaaaatttaaaaggcgcaaccaagaacgcctcaatccccgagacgtggagtatggcactatcgaagggctcatcaccaccaatgttggagacagcactttagcagatttattggatcattcgtgtacatcaggaagtggctctggtcttccttttctggtacaaagaacagtggctcgccagattacactgttggagtgtgtcgggaaaggcaggtatggtgaggtgtggaggggcagctggcaaggggagaatgttgccgtgaagatcttctcctcccgtgatgagaagtcatggttcagggaaacggaattgtacaacactgtgatgctgaggcatgaaaatatcttaggtttcattgcttcagacatgacatcaagacactccagtacccagctgtggttaattacacattatcatgaaatgggatcgttgtacgactatcttcagcttactactctggatacagttagctgccttcgaatagtgctgtccatagctagtggtcttgcacatttgcacatagagatatttgggacccaagggaaaccagccattgcccatcgagatttaaagagcaaaaatattctggttaagaagaatggacagtgttgcatagcagatttgggcctggcagtcatgcattcccagagcaccaatcagcttgatgtggggaacaatccccgtgtgggcaccaagcgctacatggcccccgaagttctagatgaaaccatccaggtggattgtttcgattcttataaaagggtcgatatttgggcctttggacttgttttgtgggaagtggccaggcggatggtgagcaatggtatagtggaggattacaagccaccgttctacgatgtggttcccaatgacccaagttttgaagatatgaggaaggtagtctgtgtggatcaacaaaggccaaacatacccaacagatggttctcagacccgacattaacctctctggccaagctaatgaaagaatgctggtatcaaaatccatccgcaagactcacagcactgcgtatcaaaaagactttgaccaaaattgataattccctcgacaaattgaaaactgactgttga

SEQ ID NO: 2 - DNA, 3501 bp, Homo sapiens
atgctgctcactcttatcattctgttgccagtagtttcaaaatttagttttgttagtctctcagcaccgcagcactggagctgtcctgaaggtactctcgcaggaaatgggaattctacttgtgtgggtcctgcacccttcttaattttctcccatggaaatagtatctttaggattgacacagaaggaaccaattatgagcaattggtggtggatgctggtgtctcagtgatcatggattttcattataatgagaaaagaatctattgggtggatttagaaagacaacttttgcaaagagtttttctgaatgggtcaaggcaagagagagtatgtaatatagagaaaaatgtttctggaatggcaataaattggataaatgaagaagttatttggtcaaatcaacaggaaggaatcattacagtaacagatatgaaaggaaataattcccacattcttttaagtgctttaaaatatcctgcaaatgtagcagttgatccagtagaaaggtttatattttggtcttcagaggtggctggaagcctttatagagcagatctcgatggtgtgggagtgaaggctctgttggagacatcagagaaaataacagctgtgtcattggatgtgcttgataagcggctgttttggattcagtacaacagagaaggaagcaattctcttatttgctcctgtgattatgatggaggttctgtccacattagtaaacatccaacacagcataatttgtttgcaatgtccctttttggtgaccgtatcttctattcaacatggaaaatgaagacaatttggatagccaacaaacacactggaaaggacatggttagaattaacctccattcatcatttgtaccacttggtgaactgaaagtagtgcatccacttgcacaacccaaggcagaagatgacacttgggagcctgagcagaaactttgcaaattgaggaaaggaaactgcagcagcactgtgtgtgggcaagacctccagtcacacttgtgcatgtgtgcagagggatacgccctaagtcgagaccggaagtactgtgaagatgttaatgaatgtgctttttggaatcatggctgtactcttgggtgtaaaaacacccctggatcctattactgcacgtgccctgtaggatttgttctgcttcctgatgggaaacgatgtcatcaacttgtttcctgtccacgcaatgtgtctgaatgcagccatgactgtgttctgacatcagaaggtcccttatgtttctgtcctgaaggctcagtgcttgagagagatgggaaaacatgtagcggttgttcctcacccgataatggtggatgtagccagctctgcgttcctcttagcccagtatcctgggaatgtgattgctttcctgggtatgacctacaactggatgaaaaaagctgtgcagcttcaggaccacaaccatttttgctgtttgccaattctcaagatattcgacacatgcattttgatggaacagactatggaactctgctcagccagcagatgggaatggtttatgccctagatcatgaccctgtggaaaataagatatactttgcccatacagccctgaagtggatagagagagctaatatggatggttcccagcgagaaaggcttattgaggaaggagtagatgtgccagaaggtcttgctgtggactggattggccgtagattctattggacagacagagggaaatctctgattggaaggagtgatttaaatgggaaacgttccaaaataatcactaaggagaacatctctcaaccacgaggaattgctgttcatccaatggccaagagattattctggactgatacagggattaatccacgaattgaaagttcttccctccaaggccttggccgtctggttatagccagctctgatctaatctggcccagtggaataacgattgacttcttaactgacaagttgtactggtgcgatgccaagcagtctgtgattgaaatggccaatctggatggttcaaaacgccgaagacttacccagaatgatgtaggtcacccatttgctgtagcagtgtttgaggattatgtgtggttctcagattgggctatgccatcagtaatgagagtaaacaagaggactggcaaagatagagtacgtctccaaggcagcatgctgaagccctcatcactggttgtggttcatccattggcaaaaccaggagcagatccctgcttatatcaaaacggaggctgtgaacatatttgcaaaaagaggcttggaactgcttggtgttcgtgtcgtgaaggttttatgaaagcctcagatgggaaaacgtgtctggctctggatggtcatcagctgttggcaggtggtgaagttgatctaaagaaccaagtaacaccattggacatcttgtccaagactagagtgtcagaagataacattacagaatctcaacacatgctagtggctgaaatcatggtgtcagatcaagatgactgtgctcctgtgggatgcagcatgtatgctcggtgtatttcagagggagaggatgccacatgtcagtgtttgaaaggatttgctggggatggaaaactatgttctgatatagatgaatgtgagatgggtgtcccagtgtgcccccctgcctcctccaagtgcatcaacaccgaaggtggttatgtctgccggtgctcagaaggctaccaaggagatgggattcactgtcttgactctactccaccccctcacctcagggaagatgaccaccactattccgtaagaaatagtgactctgaatgtcccctgtcccacgatgggtactgcctccatgatggtgtgtgcatgtatattgaagcattggacaagtatgcatgcaactgtgttgttggctacatcggggagcgatgtcagtaccgagacctgaagtggtgggaactgcgccacgctggccacgggcagcagcagaaggtcatcgtggtggctgtctgcgtggtggtgcttgtcatgctgctcctcctgagcctgtggggggcccactactacaggactcagaagctgctatcgaaaaacccaaagaatccttatgaggagtcgagcagagatgtgaggagtcgcaggcctgctgacactgaggatgggatgtcctcttgccctcaaccttggtttgtggttataaaagaacaccaagacctcaagaatgggggtcaaccagtggctggtgaggatggccaggcagcagatgggtcaatgcaaccaacttcatggaggcaggagccccagttatgtggaatgggcacagagcaaggctgctggattccagtatccagtgataagggctcctgtccccaggtaatggagcgaagctttcatatgccctcctatgggacacagacccttgaagggggtgtcgagaagccccattctctcctatcagctaacccattatggcaacaaagggccctggacccaccacaccaaatggagctgactcagtga

SEQ ID NO: 3 - RNA, 110 nt, Homo sapiens
acccggcagugccuccaggcgcagggcagccccugcccaccgcacacugcgcugccccagacccacugugcgugugacagcggcugaucugugccugggcagcgcgaccc

SEQ ID NO: 4 - RNA, 21 nt, Homo sapiens
ucagugcaugacagaacuugg

SEQ ID NO: 5 - DNA, 1380 bp, Artificial Sequence
  Cloning Vector
tagttattactagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagct

(19) United States
(12) Patent Application Publication
(10) Pub. No.: US 2018/0344448 A1
(43) Pub. Date: Dec. 6, 2018

(54) TRANSFECTED EPIDERMAL GRAFTS AND METHODS OF MAKING THE SAME

(71) Applicant: KCI LICENSING, INC., San Antonio, TX (US)

(72) Inventors: Sandra OSBORNE GARCIA, San Antonio, TX (US); John R. HARPER, Boerne, TX (US)

(21) Appl. No.: 15/772,507

(22) PCT Filed: Feb. 17, 2017
(86) PCT No.: PCT/US2017/018431

§ 371 Date: Apr. 30, 2018

Related U.S. Application Data
(60) Provisional application No. 62/297,340, filed on Feb. 19, 2016.

(51) Int. Cl. A61F 2/10; A61L 27/36; A61L 27/60

(52) U.S. Cl. CPC A61F 2/105; A61L 27/362; A61F 2240/001; A61L 2300/258; A61L 2300/412; A61L 27/60

(57) ABSTRACT

The present application relates to devices and methods for harvesting a skin graft(s) and introducing one or more nucleic acid, one or more amino acid sequences, or a combination thereof into one or more cells of the skin graft. The present invention provides transfecting the skin graft to modulate a cellular response that enhances epithelialization and/or pigmentation.

### RELATED APPLICATION

The present application claims priority to provisional patent application No. 62/297,340, filed Feb. 19, 2016. This provisional application is herein incorporated by reference in its entirety.

### BACKGROUND

Skin is the largest organ of the human body, representing approximately 16% of a person's total body weight. Because it interfaces with the environment, skin has an important function in body defense, acting as an anatomical barrier from pathogens and other environmental substances. Skin also provides a semi-permeable barrier that prevents excessive fluid loss while ensuring that essential nutrients are not washed out of the body. Other functions of skin include insulation, temperature regulation, and sensation. Skin tissue may be subject to many forms of damage, including burns, trauma, disease, and depigmentation (e.g., vitiligo, postburn dyspigmentation, piebaldness, idiopathic guttate hypomelanosis, discoid lupus erythematosus leukoderma).

Skin grafts are often used to repair such skin damage. Skin grafting is a surgical procedure in which a section of skin is removed from one area of a person's body (autograft), removed from another human source (allograft), or removed from another animal (xenograft), and transplanted to a recipient site of a patient, such as a wound site. As with any surgical procedure, skin grafting includes certain risks. Complications may include graft failure, rejection of the skin graft, infections at donor or recipient sites, or autograft donor sites oozing fluid and blood as they heal. Some of these complications (e.g., graft failure and rejection of the skin graft) may be mitigated by using an autograft instead of an allograft or a xenograft.

A problem encountered when using an autograft is that skin is taken from another area of a person's body to produce the graft, resulting in trauma and wound generation at the donor site. Generally, the size of the graft matches the size of the recipient site, and thus a large recipient site requires removal of a large section of skin from a donor site. As the size of the section of skin removed from the donor site increases, so does the probability that the donor site will not heal properly, requiring additional treatment and intervention. Additionally, as the size of the section of skin removed from the donor site increases, so does the possibility of infection. There is also increased healing time associated with removal of larger sections of skin because a larger wound is produced.

Moreover, autologous skin grafts can be compromised if the donor is aged or unhealthy. Skin grafts from elderly patients and individuals with underlying health problems often exhibit slower re-epithelialization rates and/or greater chance of infections thereby compromising the integrity of the graft and/or making it unsuitable for use.

There is an unmet need for enhancing a cellular response of cells in skin grafts.

### SUMMARY

The present invention provides systems and methods of transfecting skin graft (e.g., epidermal) cells to achieve a desired cellular response. One or more nucleic acid sequences and/or one or more amino acid sequences can be introduced into some or all of the cells comprising an epidermal skin graft, e.g., cells harvested for a skin graft. Also disclosed herein are methods of treating or modulating epidermal metabolic disorders or reducing wound closure time using the enhanced skin, or otherwise enhancing skin grafts cells due to age, health conditions, etc. The epidermal cells can be visualized during the epithelialization process to monitor wound closure.

In one embodiment, the present invention relates to a skin graft (e.g., a micrograft) comprising an outer stratum corneum layer and an inner basal layer comprising one or more cells and/or one or more cell layers, wherein at least one (e.g., some or all) of the one or more cells comprises at least one exogenous nucleic acid and/or amino acid sequence, and wherein the at least one exogenous sequence modulates a cellular response that promotes an enhanced epithelialization and/or pigmentation rate. In some embodiments, the at least one exogenous sequence further comprises a fluorescent marker (e.g., Green Fluorescent Protein and variants) for visualizing and/or monitoring epidermal cell migration and closure.

In another embodiment, the present invention relates to a skin graft harvesting system comprising a device comprising a hollow body, the hollow body having a distal end configured for placement on skin, the body further adapted to be coupled to a vacuum source, such that a negative pressure can be generated within the device when the body is placed on a donor's skin to raise at least one blister; a harvesting member integrated in said body for cutting said blister produced on said skin, the harvesting member comprising a bottom plate, a cutter plate and a top plate, each plate having at least one hole, said holes forming an aligned hole array such that a raised skin blister can be pulled through the holes in each of the plates by the negative pressure, the harvesting member further comprising an actuator for moving the cutter plate to disrupt the alignment of holes and cut the raised blister.

The skin harvesting system further includes a transfection station, wherein the transfection station is configured to receive and transfect at least one exogenous nucleic acid sequence and/or at least one amino acid sequence into a harvested skin graft, and wherein the at least one exogenous nucleic acid and/or amino acid sequence modulates a cellular response that promotes an enhanced epithelialization and/or pigmentation rate.

In another embodiment, the present invention relates to methods of treating a skin wound by harvesting a skin graft comprising one or more cells from a donor site; introducing at least one nucleic acid and/or amino acid sequence into the one or more cells of said skin graft, thereby producing a transfected skin graft, wherein the at least one nucleic acid sequence modulates a cellular response that promotes an enhanced epithelialization and/or pigmentation rate; and grafting said transfected skin graft to the skin wound, thereby treating the skin wound.

In the methods of genetically modifying a skin graft described herein, the methods can comprise obtaining an autologous skin graft from a donor site on an individual and introducing a nucleic acid and/or an amino acid sequence into the one or more cells of said skin graft, thereby producing a transfected skin graft, wherein the nucleic acid and/or amino acid sequence modulates a cellular response thereby enhancing the epithelialization rate.

In another embodiment, a method of modulating a cellular response in a skin graft comprises harvesting a skin graft from a donor site and introducing at least one nucleic acid sequence into one or more cells of said skin graft ex vivo, thereby producing a transfected skin graft, wherein the modulation of the cellular response is an epithelialization rate and/or a pigmentation rate of the one or more cells.

The methods of the present invention can further include transferring the harvested skin graft onto a first substrate. In some embodiments, the methods further include transferring the skin graft from the first substrate to a second substrate. In some embodiments, the first and second substrates are the same material. In other embodiments, the first and second substrates are different materials. For example, the first and second substrates can be medical dressings. The orientation of the skin graft can be maintained while transferring said graft from the first substrate to the second substrate.

The methods of the present invention can further comprise enhancing wound closure (i.e., decreasing wound closure time) after grafting said transfected skin graft to the skin wound on the individual.

In another embodiment, the invention relates to a method of monitoring wound closure. The method can comprise obtaining an autologous skin graft from a donor site on an individual, introducing a nucleic acid sequence into the one or more cells of said skin graft ex vivo, thereby producing a transfected skin graft, wherein the nucleic acid sequence comprises a fluorescence coding sequence, and detecting fluorescence to provide an indication of wound closure progress.

In the skin grafts, skin graft harvesting systems and methods described herein, the at least one nucleic acid sequence can comprise a DNA sequence, a RNA sequence, or a combination thereof. In some embodiments, the nucleic acid sequence can comprise any nucleic acid suitable for introduction into one or more cells (e.g., by transfection). For example, the nucleic acid sequence can comprise a circular DNA sequence (e.g., a plasmid). In some embodiments, the at least one nucleic acid sequence is an exogenous sequence. In other embodiments, the at least one nucleic acid sequence is an endogenous sequence.

For example, the at least one nucleic acid sequence can be an activin sequence, an antisense-miRNA sequence, a microRNA family sequence, a β-nerve growth factor sequence, a chemokine sequence, an epidermal growth factor sequence, a fibroblast growth factor sequence, a hepatocyte growth factor sequence, an insulin-like growth factor sequence, an interleukin sequence, a keratinocyte growth factor 1 sequence, a neuregulin sequence, a platelet derived growth factor sequence, a transforming growth factor α sequence, a transforming growth factor β1 or β2 sequence, a vascular endothelial growth factor sequence, a β-3,4-dihydroxyphenylalanine (DOPA) sequence, a melanogenesis producing gene (e.g., a monophenol monooxygenase sequence, a 3,4-β-dihydroxyphenylalanine oxygen oxidoreductase sequence, a tyrosinase-related protein 1 (TYRP1) sequence, a DOPAchrome tautomerase (DCT) sequence), an endothelin-1 (ET-1) sequence, a proopiomelanocortin (POMC) sequence, a melanocyte-stimulating hormone (MSH) sequence, a fluorescence coding sequence (e.g., green fluorescent protein (GFP) and variants of GFP such as, yellow fluorescent protein, cyan fluorescent protein, blue fluorescent protein or red fluorescent protein), or a combination thereof.

The at least one exogenous sequence can also comprise a fluorescent marker sequence. Fluorescent markers can allow for the visualization of and/or monitoring of epidermal cell migration and/or wound closure. Types of fluorescent tags or markers can include, for example, GFP and variants of GFP, such as YFP, BFP and CFP, or any fluorophore or fluorochrome.

In some embodiments, the nucleic acid sequences and/or the amino acid sequences are introduced into one or more cells by a transfection method. The transfection method can be a chemical transfection, a non-chemical transfection technique, or a combination of both. For example, the chemical transfection can be selected from the group consisting of calcium phosphate transfection, lipofection (e.g., liposome-mediated transfection, cationic lipid transfection), cationic polymer transfection (e.g., DEAE-dextran mediated transfection) and cationic amino acid transfection. Alternatively, a non-chemical transfection can be selected from the group consisting of electroporation, sonoporation, laser-mediated (e.g., optical) transfection, direct injection (e.g., microinjection), magnetofection, impalefection, biolistic particle delivery transfection, viral delivery, and receptor-mediated uptake.

The skin grafts of the present invention will typically include one or more cells (e.g., one or more layers of cells) from an epidermis. For example, the one or more cells from the epidermis can comprise an epithelial cell (e.g., an stratified squamous epithelial cell), a keratinocyte, a basal cell, a melanocyte, a Langerhans cell, a Merkel cell, an epidermal stem cell, an epithelial progenitor cell, or combinations thereof. In some embodiments, the skin graft is preferably an autologous skin graft. In some embodiments, the skin graft is an epidermal allograph or a xenograph.

### BRIEF DESCRIPTION OF THE DRAWINGS

FIG. 1 provides an exploded view of the device.

FIG. 2A-2B are schematics showing an exemplary device for generating and harvesting a plurality of micrografts. FIG. 2A provides a top view of the assembled device.

FIG. 2B provides a side view of the assembled device.

FIGS. 3A-3J provide schematics of an exemplary process for preparing and transfecting a skin graft according to methods of the invention. FIG. 3A shows an excised epidermal blister sitting on a sterile cutting surface with a sterile cutter tool above. FIG. 3B shows the cutter tool cutting the epidermal blister to generate an array of micrografts. FIG. 3C shows the array of micrografts that has been produced by the cutting tool sitting on a first substrate. FIG. 3D shows the first substrate placed into an expansion device. A second substrate is placed into the assembly cap above. FIG. 3E shows the expansion process. As the first substrate expands, the micrografts move apart. FIG. 3F shows that as the first substrate flattens against the assembly cap, the micrografts are transferred to the second substrate. FIG. 3G shows the completed expansion process and that the micrografts have been transferred to the second substrate. FIG. 3H shows removal of the assembly cap having the second substrate and expanded micrografts from the expansion device. FIG. 3I shows removal of the second substrate having the expanded micrografts from the assembly cap of the expansion device. FIG. 3J shows the array of micrografts of FIG. 3C in a transfection station.

FIG. 4 is a schematic, perspective top view of a skin graft harvester for use with the substrate.

FIG. 5 is a schematic, perspective top view of the skin graft harvester of FIG. 4 with the head component removed and the cutter mechanism exposed.

FIG. 6 is a schematic, perspective top view of the skin graft harvester of FIG. 4 with an absorbent substrate according to the invention deployed in the harvester to capture skin grafts.

FIG. 7 is a flow diagram of embodiments of the methods of the present invention

FIGS. 8A-8C illustrate methods of transfecting a skin graft of the present invention. FIG. 8A illustrates skin graft harvesting and transfection station preparation. FIG. 8B illustrates transfection of skin graft cells in the transfection station using a chemical transfection. FIG. 8C illustrates transfection of skin graft cells in the transfection station using electroporation.

FIG. 9 illustrates further transfection methods according to the present invention.

### DETAILED DESCRIPTION

The present invention generally relates to devices, systems and methods of harvesting a skin graft and transfecting one or more cells of the skin graft with a (i.e., one or more) nucleic acid sequence and/or amino acid sequence. The device can raise a blister (e.g., a suction blister) and cut the raised blister, i.e., a blister raising device integrated with a cutting member. The present invention further provides systems and methods of integrating a skin graft device with transfecting skin cells to achieve a cellular response. One or more nucleic acid and/or amino acid sequences can be introduced into cells of an epidermal skin graft, e.g., cells harvested for a skin graft.

Also disclosed herein are methods of treating skin wounds and/or monitoring wound closure. As used herein a “skin wound” relates to any skin disorder or condition, including but not limited to epidermal disorders (e.g., metabolic skin disorders), skin depigmentation disorders (e.g., vitiligo, postburn dyspigmentation, piebaldness, idiopathic guttate hypomelanosis and discoid lupus erythematosus leukoderma), burns, cuts and trauma, and other skin ailments. Also disclosed herein are methods of visualizing and/or monitoring reepithelialization (e.g., of wounds) and/or repigmentation.

In certain embodiments, devices of the invention are configured to produce epidermal grafts. The skin consists of 2 layers. The outer layer, or epidermis, is derived from ectoderm, and the thicker inner layer, or dermis, is derived from mesoderm. The epidermis constitutes about 5% of the skin, and the remaining 95% is dermis. The skin varies in thickness depending on anatomic location, gender, and age of the individual. The epidermis, the more external of the two layers, is a stratified epithelium consisting primarily of melanocytes and keratinocytes in progressive stages of differentiation from deeper to more superficial layers. The epidermis has no blood vessels; thus, it must receive nutrients by diffusion from the underlying dermis through the basement membrane, which separates the 2 layers.

The dermis is a more complex structure. It is composed of 2 layers, the more superficial papillary dermis and the deeper reticular dermis. The papillary dermis is thinner, including loose connective tissue that contains capillaries, elastic fibers, reticular fibers, and some collagen. The reticular dermis includes a thicker layer of dense connective tissue containing larger blood vessels, closely interlaced elastic fibers, and coarse, branching collagen fibers arranged in layers parallel to the surface. The reticular layer also contains fibroblasts, mast cells, nerve endings, sensory organs, lymphatics, and some epidermal appendages. Surrounding the components of the dermis is the gel-like ground substance composed of mucopolysaccharides (primarily hyaluronic acid), chondroitin sulfates, and glycoproteins.

In a graft, the characteristics of the donor site are more likely to be maintained after grafting to a recipient site as a function of the thickness of the dermal component of the graft. However, thicker grafts require more favorable conditions for survival due to the requirement for increased revascularization. It has been discovered, however, that a substantially epidermal graft according to the invention is more likely to adapt to the characteristics of the recipient site.

An epidermal graft refers to a graft that consists of substantially epidermis and does not include any substantial portion of the dermal layer. A split thickness graft refers to a graft that includes sheets of superficial (epithelial) and some portion of the deep layers (dermal) of skin. A full-thickness graft refers to a graft that includes all of the layers of the skin including blood vessels and hair follicles.

Devices of the invention may be used to harvest a (one or more) skin graft for repair of numerous different types of skin damage. For example, harvested grafts may be used to treat skin wound, including but not limited to burns (e.g., both thermal and chemical burns), blistering, dermatological conditions (e.g., epidermolysis bullosa or pyoderma gangrenosum), radiation therapy ulcers, diabetic ulcers, ischemic ulcers, trophic ulcers, trauma, or depigmentation (e.g., vitiligo).

In particular embodiments, the skin graft(s) are used to treat vitiligo. Vitiligo is a chronic disorder that causes depigmentation of patches of skin. It occurs when melanocytes, the cells responsible for skin pigmentation, die or are unable to function. Although patches are initially small, they often enlarge and change shape. When skin lesions occur, they are most prominent on the face, hands and wrists. Some lesions have hyper-pigmentation around the edges. Depigmentation is particularly noticeable around body orifices, such as the mouth, eyes, nostrils, genitalia and umbilicus.

Vitiligo is generally classified into two categories, non-segmental vitiligo and Segmental vitiligo. In non-segmental vitiligo (NSV), there is usually some form of symmetry in the location of the patches of depigmentation. New patches also appear over time and can be generalized over large portions of the body or localized to a particular area. Vitiligo universalis is where little pigmented skin remains on the body. Non-segmental vitiligo can come about at any age, unlike segmental vitiligo which is far more prevalent in teenage years.

Segmental vitiligo (SV) differs in appearance, aetiology and prevalence from associated illnesses. Its treatment is different from that of non-segmental vitiligo. It tends to affect areas of skin that are associated with dorsal roots from the spine. It spreads much more rapidly than non-segmental vitiligo and, without treatment, it is much more stable/static in course and not associated with auto-immune diseases.

To treat vitiligo, a pigmented autograft is provided to the site of depigmented skin. The graft includes melanocytes, and thus upon the recipient site accepting the graft, the graft will produce pigmented skin at the recipient site. A donor site of pigmented skin is aseptically cleaned prior to harvesting of a skin graft. Standard methods are used to clean the donor site. A typical donor site is an inner thigh, but any area of pigmented skin may be used.

After cleaning, a skin grafted is harvested. Devices described herein raise and cut a blister(s), such as a suction blister. The recipient site is prepared through aseptic cleaning and dermabrasion. The graft(s) is applied to the dermabraded recipient site. The donor site and the recipient site are dressed and wound care is provided.

As used herein, “transfection” and “transfecting” refers to any method to introduce a nucleic acid (e.g., DNA, RNA) or an amino acid (e.g., peptides, proteins) into a cell. For instance, cells of skin grafts obtained from the donor site can be further enhanced or modified by introducing (e.g., by transfection) one or more nucleic acid sequences and/or one or more amino acid sequences into one or more cells of the skin graft. Cells of skin grafts can also be transfected with fluorescent marker, such as green fluorescent protein.

In some embodiments, the nucleic acid sequences used for transfection are exogenous sequences. In other embodiments, the nucleic acid sequences are endogenous sequences. In other embodiments, the nucleic acid sequences are a combination of exogenous and endogenous sequences. As described herein, the one or more nucleic acid sequences can, when expressed by a cell, enhance (directly or indirectly), for example, an epithelialization rate and/or repigmentation rate. The skin graft can then be placed at a recipient site (e.g., a skin wound such as a burn or cut), with enhanced epithelialization and/or repigmentation.

The present invention generally relates to methods and devices for harvesting and preparing a skin graft. In certain embodiments, methods of the invention allow for preparing a skin graft for transfer to a recipient site. The devices and methods of the invention use mechanical techniques for preparation of a skin graft.

In a skin graft, the characteristics of the donor site are more likely to be maintained after grafting to a recipient site as a function of the thickness of the dermal component of the graft. However, thicker grafts require more favorable conditions for survival due to the requirement for increased revascularization. It has been discovered, however, that a substantially epidermal graft according to the invention is more likely to adapt to the characteristics of the recipient site. Further, an epidermal graft can be further improved to adapt to the characteristics of the recipient site by introducing (e.g., by transfecting) one more nucleic acid sequences to one or more cells of the skin graft.

The present invention provides methods and devices for preparing and using skin grafts that have been transfected with one or more nucleic acids. In certain embodiments, the invention relates to introducing (i.e., transfecting) one or more nucleic acid sequences to the skin graft (i.e., cells of the graft) before transferring the skin graft to a recipient site.

In certain embodiments, methods of the invention involve harvesting a plurality of skin grafts from a subject, applying the grafts to a first substrate, transfecting one or more cells of the skin graft with one or more nucleic acid sequences, and transferring the grafts from the first substrate to a patient recipient site.

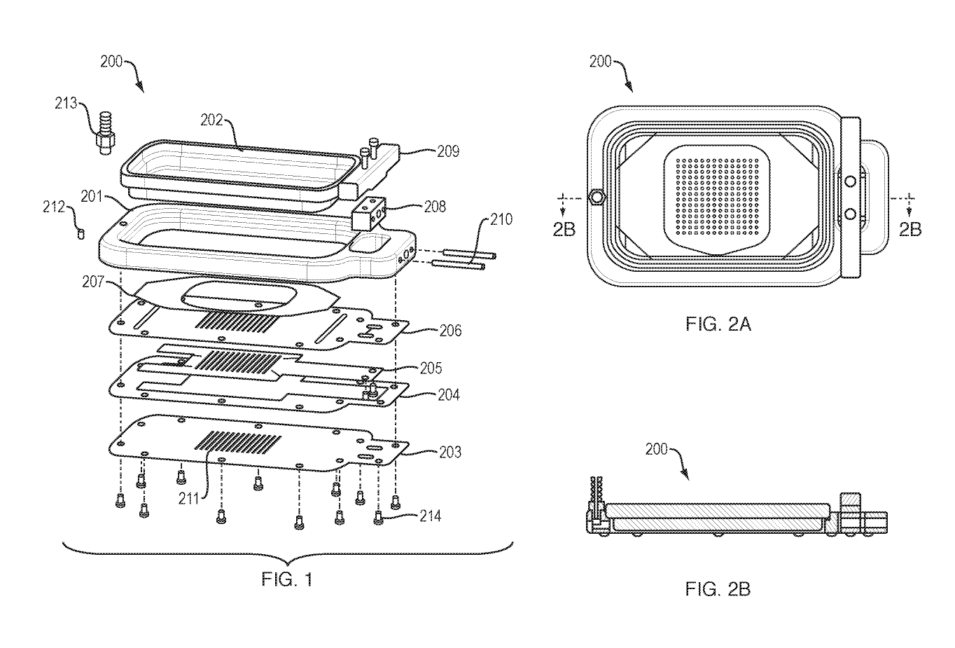

In certain embodiments, an exemplary device as shown in FIG. 1 and FIGS. 2A-2B is used to obtain the plurality of skin grafts. Device 200 includes a frame 201 and a lid 202. Fitted into the frame is a bottom plate 203, a cutter grid plate 204, a cutter plate 205, and a top plate 206. The bottom plate 203, the cutter plate 205, and the top plate 206, each include a hole array 211. Once assembled, the hole array 211 of each of plates 203, 205, and 206 are aligned. The size of the holes in the hole array will depend on the size of the graft needed, with larger holes being used to produce larger grafts. A first substrate 207 interacts with the top plate 206 and will receive the harvested grafts.

Device 200 further includes an actuation block 208, actuation bar 209, and actuation block guides 210. Actuation components 208, 209, and 210 control movement of the cutter plate 205. The frame 201 includes a vacuum stop 212 and the lid 202 includes a suction hole barb 213. Once assembled, the frame 201 and lid 202 are arranged such that the vacuum stop 212 and the suction hole barb 213 are aligned with each other (FIG. 1 panel B). A vacuum source is then connected to the device 200 such that negative pressure can be generated within the device. The device 200 can be held together by clamp screws 214. Device 200 may also include a heating element.

To produce and harvest the plurality of skin grafts, device 200 is placed on a donor site, such as an inner thigh of a patient. The vacuum source is turned on, producing negative pressure within device 200. The negative pressure causes the skin to be pulled toward lid 202, with a plurality of different portions of skin being pulled through each hole array 211 in each of plates 203, 205, and 206. Such action results in generation of many microblisters. The blisters may or may not be fluid-filled. Any type of raised blister may be used with methods of the invention.

Once the microblisters are raised, actuation components 208, 209, and 210 are engaged to move cutter plate 205. The movement of cutter plate 205 disrupts the alignment of the hole arrays 211 in each of plates 203, 205, and 206, and results in cutting of the microblisters. The cut microblisters are captured on the first substrate 207 that is above top plate 206. In this manner, there is provided a spaced apart array of micrografts. The amount of negative pressure applied, the amount of time the vacuum is maintained, and/or the depth of the holes above the cutting surface (plate 206) determines what type of graft will be harvested, e.g., epidermal graft, split thickness graft, or full thickness graft. Generally, each micrograft will have a lateral dimension of less than about 2 mm e.g., 100 to 2000 microns.

Additional details on harvesters useful in connection with the present invention can be found in U.S. patent application Ser. No. 13/839,518 filed Mar. 15, 2013; U.S. patent application Ser. No. 13/346,329 filed Jan. 9, 2012 (now U.S. Pat. No. 8,978,234); U.S. patent application Ser. No. 13/436,318 also filed Jan. 9, 2012; U.S. patent application Ser. No. 13/014,737 filed Jan. 27, 2011; U.S. patent application Ser. No. 12/851,656 filed Aug. 6, 2010 (now U.S. Pat. No. 8,562,626); U.S. patent application Ser. No. 12/851,621 filed Aug. 6, 2010; U.S. patent application Ser. No. 12/851,703 filed Aug. 6, 2010 (now U.S. Pat. No. 8,926,631); and U.S. patent application Ser. No. 12/851,682 filed Aug. 6, 2010 (now U.S. Pat. No. 8,617,181). The contents of each of the above-referenced related applications are herein incorporated by reference in their entireties.

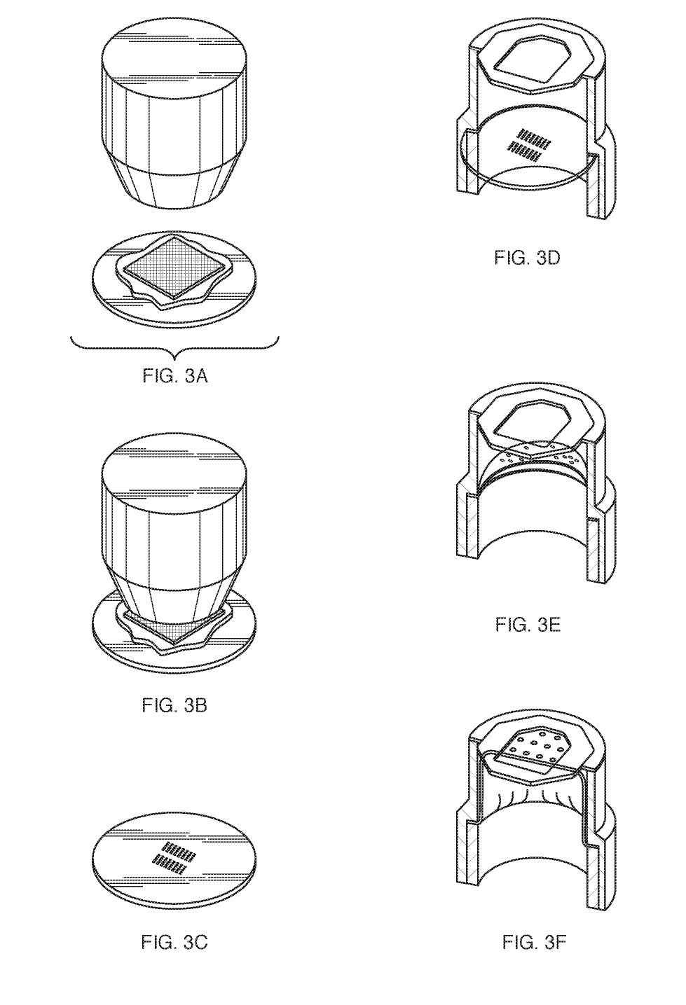

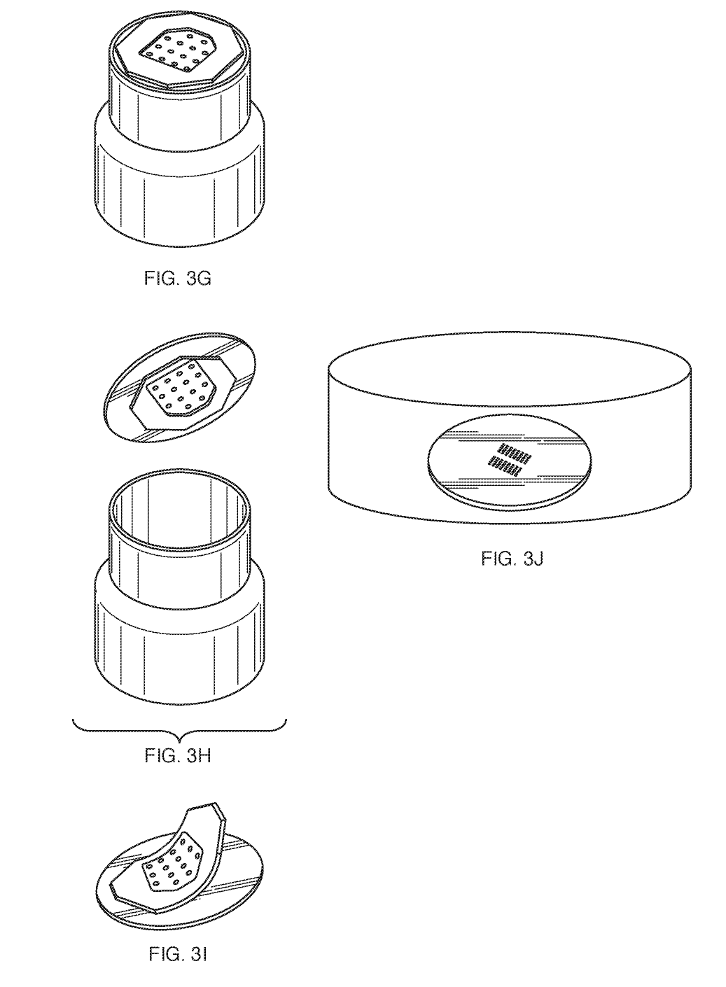

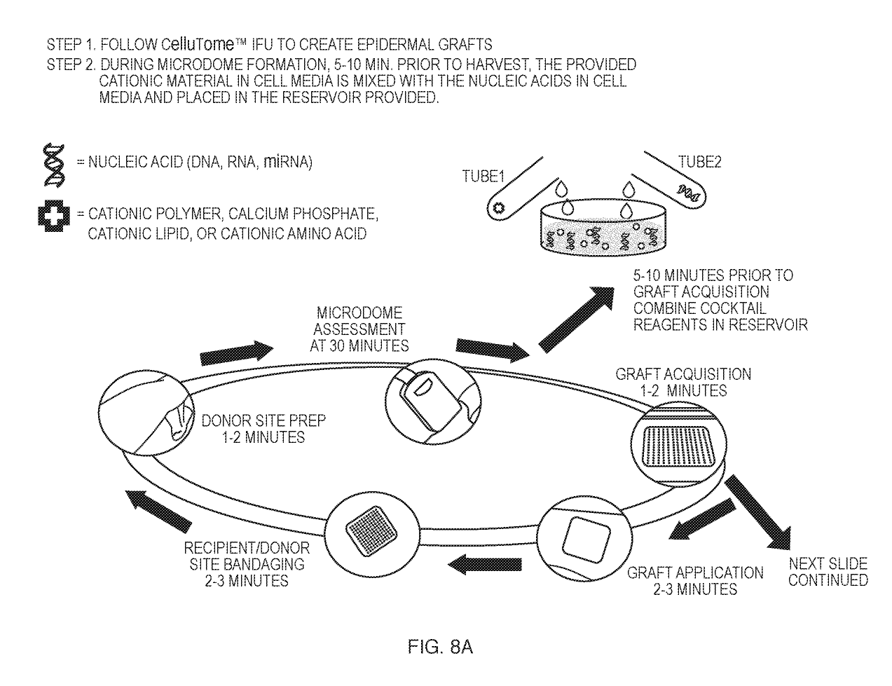

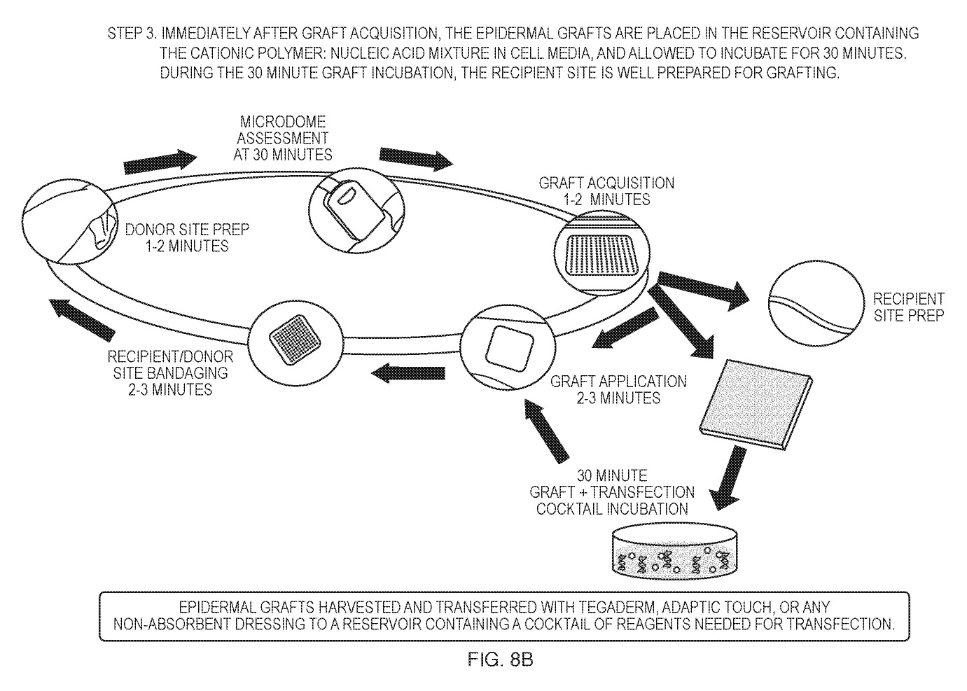

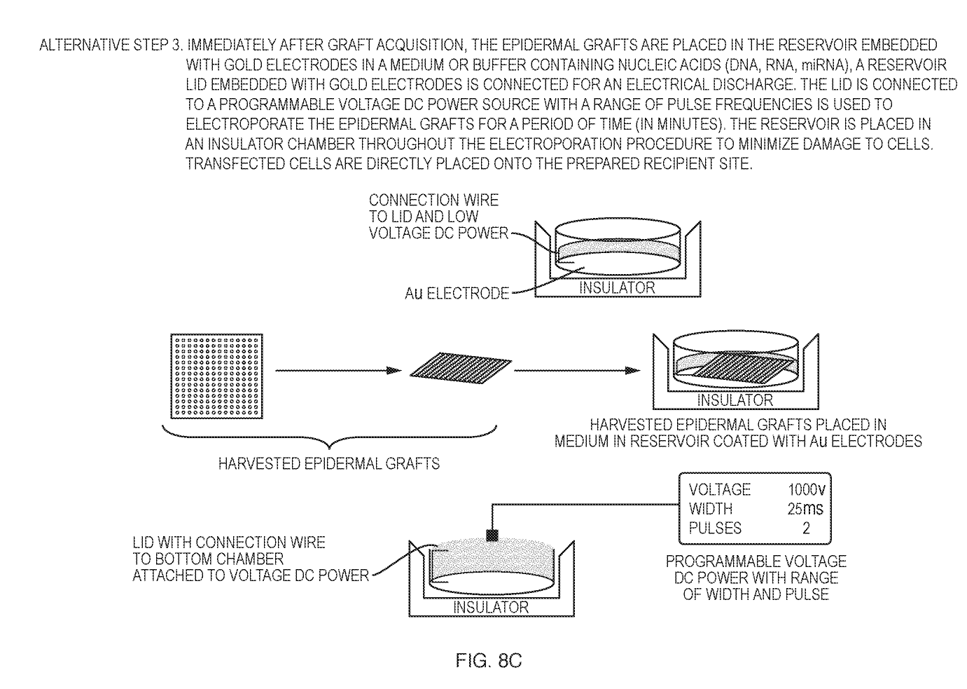

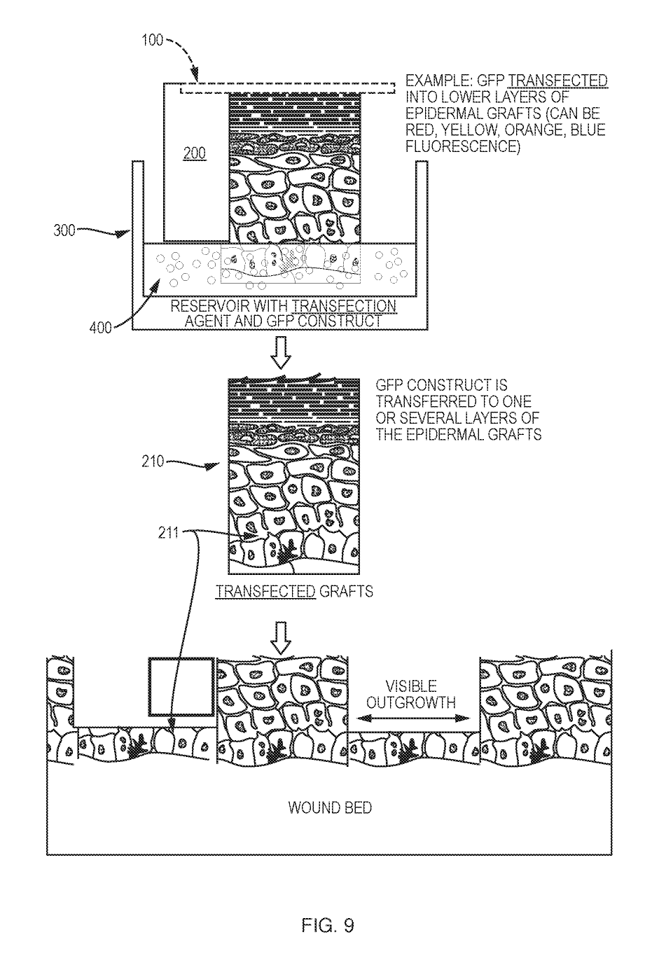

Once the grafts have been harvested and applied to the first substrate, the first substrate is placed into a reservoir for transfection (e.g., a transfection station). There, one or more cells of the grafts are transfected with one or more nucleic acid sequences. See FIGS. 3C, 3J and 8B. The grafts are oriented on the substrate so that one or more cells can be transfected with an exogenous nucleic acid sequence. For instance, the grafts are oriented such that the basal layer is facing away from the substrate layer. In this orientation, the stratum corneum layer (the outer most layer of the epidermis) is in contact with the substrate. The basal layer of the skin grafts are now in direct contact with transfection reagents in the transfection station as illustrated in FIGS. 8B-8C and 9. For example, the basal layer of skin graft 200 in FIG. 9 in transfection station 300 is in direct contact with the transfection reagents. Substrate 100 is in contact with the stratum corneum layer (outer most layer of epidermis).

In some embodiments, before the grafts are transfected with one or more nucleic acid sequences, the grafts can be stretched or expanded, resulting in increased distance between the individual micrografts, moving them apart and resulting in production of a skin graft that can repair a recipient site that is larger than the donor site from which the grafts were obtained. In methods of the invention, the individual grafts themselves are not expanded, i.e., the graft tissue is not stretched; rather, stretching of the substrate increases the space or distance between each individual micrograft. Methods of the invention thus minimize tissue manipulation.

The purpose of such processing is to use tissue from a donor site to cover a wound area that is larger than the donor site. The stretching of the substrate may be done manually, i.e., by hand, or may be done with the help of a machine. The stretching may be substantially uniform in all directions or may be biased in a certain direction. In a particular embodiment, the stretching is substantially uniform in all directions. Stretching of the substrate may be performed mechanically or may be accomplished by application of a pressurized fluid or gas. In certain embodiments, air pressure is used to expand the first substrate. Exemplary devices and methods are described in Korman (U.S. Pat. No. 5,914,264), the content of which is incorporated by reference herein in its entirety.

Any minimum distance can be provided between micrografts after the first substrate is stretched. The amount of stretching can be large enough to provide a sufficiently large area of substrate containing micrografts to allow a larger area of damaged tissue to be repaired using a particular amount of graft tissue removed from the donor site, i.e., the area of the stretched first substrate containing the separated micrografts can be much larger than the total area of the donor site. For example, the distance between adjacent micrografts on the stretched first substrate can be greater than about 0.5 mm, although small separation distances may also be used. For repigmentation of skin tissue, an amount of stretching can be applied to the first substrate such that the distance between adjacent micrografts is less than about 4 mm, because it is known that melanocytes, when grafted to a depigmented region, can migrate up to about 2 mm from each micrograft to repigment regions between the micrografts. This average distance can be larger if keratinocyte migration is involved with the tissue being treated because keratinocytes typically migrate greater distances compared to melanocytes.

The ratio of the wound area to the donor site area is referred to as the expansion ratio. A higher expansion ratio is desirable to minimize the trauma of the donor site, and to aid patients who have only a small amount of tissue available for grafting purposes. The amount of area expansion, e.g., the ratio of an area of damaged tissue that can be repaired compared to an area of graft tissue removed from a donor site, may be 500× or more. In particular embodiments, the area of expansion may be from about 10× to about 100×, which provides a more uniform coverage and/or repigmentation of the recipient site. For repairing burns or ulcerated tissue, the micrografts may be smaller than those used to repair other types of damaged tissue, and thus the distances between adjacent micrografts may be greater after stretching of the first substrate. In such an exemplary application, an area expansion of about 1000× or more may be used.

In other embodiments and depending on the material of the first substrate, maintaining the first substrate in a stretched configuration may result in stress on the substrate that is not optimal. Additionally, the stretched first substrate may not retain the same properties as the unstretched configuration of the first substrate, i.e., technological characteristics, such as physical, environmental and performance characteristics could be affected by the stretching of the substrate. Additionally, methods used to maintain the substrate in its stretched condition may be physically cumbersome and prevent uniform application of the micrografts to uneven skin surfaces. Thus in certain embodiments, once the first substrate has been stretched, the spaced apart micrografts are transferred to a second substrate. By transferring the micrografts to a second substrate, methods of the invention minimize manipulation and stress of the substrate that holds the graft to the recipient site.

After stretching the first substrate, the second substrate is brought into contact with the grafts on the stretched first substrate. Transfer is facilitated by the second substrate having greater affinity or more adhesive force toward the micrografts than the first substrate. In certain embodiments, the second substrate is coated with a hydrocolloid gel. In other embodiments, the first substrate is wetted with a fluid such as water or a saline solution. Wetting the micrografts and the first substrate provides lubrication between the grafts and the first substrate and allows for easy transfer of the grafts from the first substrate to the second substrate. After wetting the first substrate, the grafts have greater affinity for the second substrate than the first substrate. The wetted first substrate is then removed from the second substrate and the grafts remain attached to the second substrate. The distance between the micrografts is maintained after transfer of the micrografts from the stretched first substrate to the second substrate.

After transferring the grafts from the first substrate to the second substrate, one or more nucleic acid sequences can then be introduced into one or more cells of the grafts. Alternatively, once the grafts are transferred to the first substrate, one or more nucleic acid sequences can be introduced into one or more cells of the grafts. After introduction of the one or more nucleic acid sequences, the grafts can be transferred to a second substrate as described herein. Cells of skin grafts that have been introduced with (e.g., transfected) one or more nucleic acid sequences are referred herein as “modified” micrografts or skin grafts or “enhanced” micrografts or skin grafts.

The first substrate may be made from any material that is biocompatible and capable of being stretched upon application of a moderate tensile force. The second substrate may be made from any material known in the art that is compatible with biological tissue. The second substrate may also be capable of being stretched upon application of a moderate tensile force. Exemplary materials for the first and/or second substrates include medical dressings, such as TEGADERM™ (medical dressing, commercially available from 3M, St. Paul, Minn.) or DUODERM™ (medical dressing, commercially available from 3M, St. Paul, Minn.). The first and/or second substrates may also be gas permeable.

In certain embodiments, the first and/or second substrates include an adhesive on one side that facilitates attachment of the grafts to the substrates. The substrate material may have intrinsic adhesive properties, or alternatively, a side of the substrate may be treated with an adhesive material, e.g., an adhesive spray such as LEUKOSPRAY (Beiersdoerf GmbH, Germany). In certain embodiments, the first and second substrates are the same material. In other embodiments, the first and second substrates are different materials. In certain embodiments, the materials of the first and second substrates are chosen to facilitate transfer of the micrografts from the first substrate to the second substrate. For example, in certain embodiments, the material chosen for the first substrate has a weaker adhesive than the material chosen for the second substrate.

In certain embodiments, the material of the first substrate is a deformable non-resilient material. A deformable non-resilient material refers to a material that may be manipulated, e.g., stretched or expanded, from a first configuration to a second configuration, and once in the second configuration, there is no residual stress on the substrate. Such materials may be stretched to an expanded configuration without returning to their original size, and thus in these embodiments it is not necessary to transfer the micrografts from a first substrate to a second substrate. Instead, the expanded first substrate including the modified micrografts is applied to a recipient site.

Such deformable non-resilient materials tend to be soft, stiff or both soft and stiff. Softness is measured on the durometer scale. An example of such a material is a soft polyurethane. A soft polyurethane is produced is as follows. Polyurethanes in general usually have soft and hard segments. The hard segments are due to the presence of phenyl bridges. In a soft polyurethane, the phenyl bridge is switched out for an aliphatic, which is more flexible as its 6 carbon ring has no double bonds. Therefore, all the segments are soft. On the Durometer Scale, a soft polyethylene is rated about Shore 80 A. Other materials suitable for use with methods of the invention include low density polyethylene, linear low density polyethylene, polyester copolymers, polyamide copolymers, and certain silicones. In these embodiments, the expanded first substrate having the micrografts retains its expanded position without any residual stress, and the expanded first substrate is applied to a recipient site.

In some embodiments, before the grafts are transfected with one or more nucleic acid sequences, the grafts can be disaggregated and/or digested, using known techniques in the art. This results in a population of individual, isolated cells from the epidermal skin grafts. The cells can be stem cells, basal cells, Langerhans cells, Merkel cells, epidermal stem cells, epithelial cells, keratinocytes, epidermal cells, melanocytes, epithelial progenitor cells or combinations thereof. The individual cells can then be transfected with one or more exogenous nucleic acid or amino acid sequences in the transfection station using the methods described herein. Additionally, before transfection of the cells, the cells can be separated to one or more cell types. These cells can be grown and expanded, in vitro, and then used for transfection. After transfection, the individual cells can be used to treat skin wounds and/or monitor skin wound healing.

Ultimately, the transfected skin grafts (i.e., cells) and substrate are applied to a recipient of site of a patient. Prior to applying the transfected grafts to the recipient site, the site is prepared to receive the grafts using any technique known in the art. Necrotic, fibrotic or avascular tissue should be removed. The technique used to prepare the site will depend on damage to the recipient site. For example, epidermal tissue, if present at the recipient site, can be removed to prepare the area for receiving the micrografts. Burned or ulcerated sites may not need removal of epidermal tissue, although some cleaning of the site or other preparation of the site may be performed. Wounds should be debrided and then allowed to granulate for several days prior to applying the graft. Some of the granulation tissue should be removed (e.g., debrided) since it has a tendency to harbor bacteria.

The size of the area at the recipient site can be about the same size as the area of the stretched first substrate having micrografts adhered thereto. This size generally will be greater than the area of the original graft tissue that was removed from the donor site to form the micrografts. The depigmented or damaged skin can be dermabraded with sandpaper or another rough material. Alternatively, the epidermal tissue can be removed from the recipient site by forming one or more blisters over the area to be treated, e.g., a suction blister or a freezing blister, and the raised epidermal blister tissue can then be removed by cutting or another procedure.

The substrate having the modified micrografts can be placed over the area to be treated to form a dressing. A portion of the substrate having the micrografts can be positioned over the area to be repaired, e.g., the area from which the epidermal tissue has been abraded or removed for repigmentation. The substrate can be fixed in place over the treatment area, e.g., using tape or the like. The substrate can be removed after sufficient time has elapsed to allow attachment and growth of the modified micrografts in the treatment area, e.g., several days to a few weeks.

Another embodiment of the invention provides harvesting a single graft from a donor site, such as an epidermal graft, generating an array of micrografts from the single graft, placing the graft on a first substrate (FIG. 3C). Optionally, the methods include expanding a distance between the micrografts on a first substrate and transferring the micrografts from the first substrate to a second substrate. The methods further include transferring the micrographs on the first substrate into a transfection station. Here, one or more nucleic acid and/or amino acid sequences are introduced into one or more cells of the graft (FIG. 3J). Finally, the modified micrografts can be applied to a recipient site. FIGS. 3A-3J provides a schematic of an exemplary process for preparing a skin graft according to methods of the invention.

Methods of the invention involve harvesting a single graft from a donor site, such as an epidermal graft. Harvesting of the skin grafts may be accomplished by any technique known in the art (e.g., suction cup(s), use of syringe with a vacuum, and harvesting with a sharp instrument). In certain embodiments, harvesting a skin graft involves raising a blister and cutting the blister. In certain embodiments, the blister may be a fluid-filled blister (e.g. a suction blister). In other embodiments, the blister is not fluid-filled. Any type of raised blister may be used with methods of the invention.

In certain embodiments, suction blister grafting is used. Suction blister grafting involves raising a blister, and then cutting off the raised blister. An exemplary suction blister grafting technique is shown in Awad, (Dermatol Surg, 34(9):1186-1193, 2008), the content of which is incorporated by reference herein in its entirety. This article also shows various devices used to form suction blisters. A suction blister device is also described in Kennedy et al. (U.S. Pat. No. 6,071,247), the content of which is incorporated by reference herein in its entirety. An exemplary device is commercially available from Electronic Diversities (Finksburg, Md.).

A device for raising a suction blister typically operates by use of suction chambers that are attached to a patient's skin. An instrument typically contains a power source, a vacuum pump, temperature controls and all related controls to operate multiple suction chambers. The suction chambers are connected to the console by a flexible connection. Each of the chambers is controlled by a preset temperature control to provide an optimal skin warming temperature. Both chambers share an adjustable common vacuum source that affects all chambers equally.

Blister formation is accomplished by attaching the suction blister device to a patient's skin. Typically hook & loop fastener straps are used to keep the device in place. The chamber heating system provides a slight warming of an orifice plate of the device, which is in direct contact with the patient's skin surface. The application of a moderate negative pressure from the instrument console, to the chamber interior, causes the patients skin to be gently drawn through the opening(s) in the orifice plate. The results are typical suction blisters, approximately the size of the opening(s) in the orifice plate. The skin and blister area is generally not damaged and patient discomfort is minimal.

The negative pressure chamber is fabricated of mostly plastic components, with two removable threaded caps. The upper cap is fitted with a clear viewing lens so that the actual blister formation can be observed. The opposite end of the chamber is fitted with a removable orifice plate that is placed on the patient's skin. Since this plate is simply threaded onto the chamber end, multiple plates with different opening patterns can be interchanged as desired.

The interior of the device is warmed and illuminated by an array of low voltage incandescent lamps. This lamp array is controlled from the instrument console temperature controller, cycling as needed, to maintain the set point temperature. The heat from these lamps is radiated and conducted to the orifice plate, which then warms the patient's skin. The chamber is connected to the console via a composite vacuum and low voltage electrical system. Quick connections are used for the vacuum and electrical system to facilitate removal and storage.

The Negative Pressure Instrument console can be a self-contained fan cooled unit which is designed to operate on 120 VAC 60 Hz power. Vacuum is supplied by an industrial quality diaphragm type vacuum pump, capable of a typical vacuum of 20 in Hg (0-65 kpa) at 0 CFM. An analog controller that is preset to 40.degree. C. provides the temperature control for each suction chamber. This provides accurate control of the orifice plate temperature. The instrument console has internal adjustments that allow the user to recalibrate the temperature setting if desired. Other temperatures can be preset if desired. The front panel includes a vacuum gauge and vacuum bleeder adjustment to regulate the vacuum to both chambers. The console front panel also contains the connections for the chamber assemblies.

Once the suction blister is raised, it is cut by methods known in the art (see e.g., Awad, Dermatol Surg, 34(9):1186-1193, 2008), and placed on the first substrate. Once on the first substrate, an array of micrografts are generated from the single graft. FIG. 3A shows an excised skin graft on a first substrate, with a sterile cutting tool above the graft. In certain embodiments, rather than being applied directly to the first substrate, the cut blister is placed onto a sterile surface, such as a glass slide, and the array of micrografts is generated on the sterile surface prior to transfer to the first substrate. In other embodiments, the cut blister is trapped between two aligned metal screens. The screens are pushed together to cut the blister into an array of micrografts. The micrografts are then pushed out of the screens and deposited onto the first substrate using an array of pushers whose size and spacing correspond to the metal screens. In certain embodiments, the cut blister is harvested directly between the two screens for generation of the array of micrografts.

In other embodiments, the cut blister is harvested directly into a shear or punch and die device for generation of micrografts. A shear or punch die includes an array of flat-faced piston-like components that fit closely into the openings in a metal screen/mesh. In this embodiment, the cut graft is harvested onto the array of pistons, and sits between the array of pistons and the screen/mesh. The screen/mesh is closed over the cut blister and force is applied to the array of pistons. The pistons push through the holes in the screen/mesh and in the process, portions of tissue are punched out from the openings of the screen/mesh and deposited on a substrate, producing an array of micrografts on a substrate. Such embodiments allow for simultaneous generation of the array of micrografts and deposition of the array of micrografts onto the substrate.

The array of micrografts can be generated by making cuts or using other protocols to form the array of micrografts from the single graft. The cuts may pass partially or completely through the graft tissue. For example, for repigmenting skin tissue, the micrografts used may have a presence of melanocytes. Accordingly, a lateral dimension of such micrografts can be between less than about 1 mm, e.g., 200 to 1000 microns. Other exemplary sizes are between 400 and 800 microns. The area of the micrografts can be between about 0.04 mm² and about 1 mm². The exemplary sizes can provide micrografts large enough such that each micrograft is likely to contain some melanocytes, yet small enough to provide a large number of micrografts from a particular piece of graft tissue, which can facilitate a significant degree of expansion on the graft site.

For treating cuts, wounds, burns or ulcers, where presence and proliferation of keratinocytes is important, the micrograft sizes may be smaller. For example, a lateral dimension of modified micrografts containing keratinocytes can be between about 50 microns and about 1000 microns, or between 100 microns and about 800 microns. The area of such modified micrografts can be between about 0.0025 mm² and about 1 mm². The exemplary size ranges provide modified micrografts large enough to contain viable and undamaged keratinocytes, and small enough to facilitate repair of a larger area of damaged skin.

FIG. 3B shows an exemplary cutting tool. The cutting tool may be configured in any manner, and such configuration will depend upon the size of the micrografts to be produced and the desired array pattern. The cutting tool includes a plurality of adjacent blades. The arrangement of the blades will depend upon the desired pattern for the array of micrografts. The tool shown in FIG. 3B is configured to produce a square grid of micrografts (See FIG. 3C). The spacing of the blades in the cutting tool will depend on the desired size of the micrografts. For example, the blades may be spaced about 100 to 2000 microns apart, or about 500 to 1000 microns apart. The cutting tool is pressed at least once into the skin graft on the first substrate to produce the array of micrografts (See FIGS. 3B-3C).

Other exemplary devices for producing an array of micrografts include mesh devices. Such mesh devices include rigid, biocompatible material, such as stainless steel. The mesh includes a plurality of openings. The openings are sized to provide an array of micrografts of a desired size, such as lateral sizes between about 100 microns and about 1000 microns or about 300 microns to about 500 microns. Similar to the cutting tool described above, the mesh is pressed at least once into the skin graft to produce the array of micrografts.

FIG. 3D-3I show optional steps of the method. Once the array of micrografts are on the first substrate, the distance between the micrografts can be expanded. Expansion results in increased distance between the individual micrografts, moving them apart and resulting in production of a skin graft that can repair a recipient site that is larger than the donor site from which the grafts were obtained. Expansion may be performed as described above. After expansion of the first substrate, the second substrate is brought into contact with the grafts on the stretched first substrate for transfer of the micrografts from the expanded first substrate to the second substrate. Transfer may be performed as described above. The distance between the micrografts is maintained after transfer of the micrografts from the stretched first substrate to the second substrate. Once the grafts have been transferred to the second substrate, the grafts and substrate can be used to introduce one or more nucleic acid sequences into the cells of the grafts. The modified grafts can then be applied to a recipient of site of a patient. Preparation of the recipient site and application of the array of modified micrografts to the prepared recipient site may be performed as described above.

In other embodiments, transfer to a second substrate is not necessary because the material of the first substrate is a deformable non-resilient material. A deformable non-resilient material refers to a material that may be manipulated, e.g., stretched or expanded, from a first configuration to a second configuration, and once in the second configuration, there is no residual stress on the substrate. Such materials may be stretched to an expanded configuration without returning to their original size. Exemplary materials are described above. In these embodiments, the expanded first substrate having the micrografts retains its expanded position without any residual stress, and the expanded first substrate is applied to a recipient site. Preparation of the recipient site and application of the array of modified micrografts to the prepared recipient site may be performed as described above.

In certain embodiments, methods of the invention maintain a proper orientation of a skin graft. Epidermal skin includes an outer stratum corneum layer and a deeper basal layer. The stratum corneum refers to the outermost layer of the epidermis, composed of large, flat, polyhedral, plate-like envelopes filled with keratin, which is made up of dead cells that have migrated up from the stratum granulosum. This layer is composed mainly of dead cells that lack nuclei. The thickness of the stratum corneum varies according to the amount of protection and/or grip required by a region of the body. In general, the stratum corneum contains 15 to 20 layers of dead cells, and has a thickness between 10 and 40 μm.

The basal layer (or stratum germinativum or stratum basale) refers to the deepest layer of the 5 layers of the epidermis. The basal layer is a continuous layer of viable cells. These cells are undifferentiated and proliferative, i.e., they create daughter cells that migrate superficially, differentiating during migration. Keratinocytes and melanocytes are found in the basal layer. Other basal layer cells can include stem cells, Langerhans cells, Merkel cells, epidermal stem cells, epithelial cells, epidermal cells, and epithelial progenitor cells.

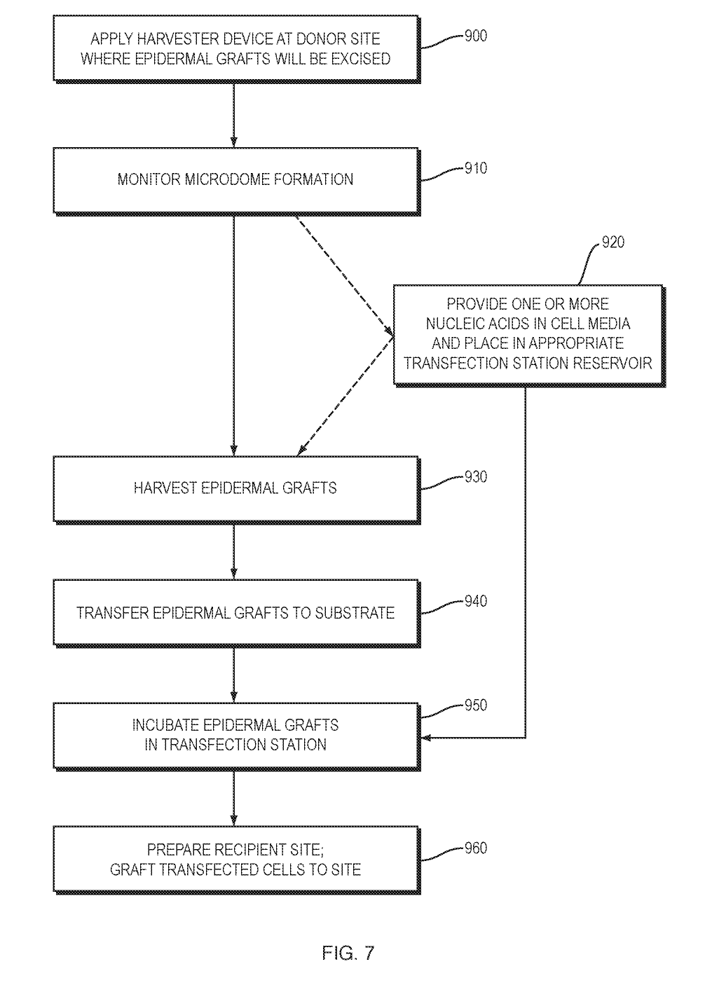

As described herein, maintenance of proper orientation of the epidermal grafts can preferentially allow for cells in the inner basal layer (stratum basale) to be transfected with one or more nucleic acid sequences and/or amino acid sequences (e.g., illustrated in FIG. 9). When attached to a substrate, the basal layer can face away from the substrate layer, i.e., the inner basal layer of the epidermal graft is not in direct contact with the substrate. Due to this orientation, the basal layer can be in direct contact with the one or more nucleic acids and other reagents during transfection. The outer stratum corneum can be in direct contact with the substrate. Maintaining proper orientation (see, e.g., step 940 in FIG. 7) of the cells (e.g., in a epidermal graft), a maximum number of cells can be transfected with one or more nucleic acids. Moreover, these cells are in direct contact with a solution comprising the one or more nucleic acids.

For example, all or some of the cells in the basal layer can be transfected with a (one or more) nucleic acid, while maintaining the integrity of the skin grafts. In some embodiments, about 10%, about 20%, about 30%, about 40%, about 60%, about 70%, about 80%, about 90% or more of the cells are transfected with one or more nucleic acids. In some embodiments, the percentage of cells transfected with the one or more nucleic acids depends on the type of nucleic acid (e.g., DNA, RNA, plasmid, etc.) and the type of cell (e.g., keratinocytes, melanocytes, stem cells, epithelial progenitor cells, etc.).

For a graft to become integrated at a recipient site, the graft must be able to receive nutrients. Since the cells of the basal layer are viable cells, orienting an epidermal graft such that the basal layer interacts with the recipient site allows the graft to receive nutrients, and thus remain viable. Additionally, the nucleic acid transfected into cells of the basal layer aids in or promotes maintaining viability, improves epithelialization, and cellular health. In contrast, since the cells of the stratum corneum are dead cells, orienting an epidermal graft such that the stratum corneum layer interacts with the recipient site prevents the graft from receiving nutrients, resulting in death of the graft tissue and graft failure. Methods of the invention ensure that during the grafting process, the basal layer of a graft interacts with the recipient site of a patient, allowing for the graft to receive nutrients and thus remain viable.

Certain methods involve harvesting an epidermal skin graft, introducing one or more nucleic acid sequences to the skin graft and applying the modified epidermal skin graft to a recipient site such that the basal layer of the skin graft makes direct contact with the recipient site. Harvesting may be accomplished by creating a blister, such as a suction blister. Suction blister grafting is described herein.

In one embodiment, a vacuum is used to hold the stratum corneum side of the blister, which can be released when the blister is deposited onto the cutting surface. In other embodiments, after the blister has been raised and prior to cutting the blister, an adhesive side of a substrate is placed in contact with the stratum corneum layer of the raised blister. Upon cutting the blister, the stratum corneum layer of the graft becomes adhered to the substrate, and the basal layer is orientated away from the substrate. Such a technique ensures that the basal layer of the graft is oriented away from the substrate and is thus available to interact with the recipient site of a patient.

Other methods of the invention involve harvesting a skin graft from a donor site, placing the skin graft on a first substrate such that basal cells of the graft make direct contact with the first substrate, transferring the graft from the first substrate to a second substrate such that the basal cells do not directly contact the second substrate, introducing one or more nucleic acid sequences to cells while the graft is in contact with the second substrate, and applying the second substrate to a recipient site. Harvesting may be accomplished by creating a blister, such as a suction blister. Suction blister grafting is described above. The blister is cut and the basal layer of the graft is contacted to an adhesive side of a first substrate. The basal layer of the graft becomes adhered to the first substrate and the stratum corneum layer is orientated away from the first substrate, and is available for interaction with a second substrate.

An adhesive side of a second substrate is brought into contact with the stratum corneum layer of the graft that is adhered to the first substrate. Transfer to the second substrate is accomplished as described above. Briefly, in one embodiment, the first substrate is wetted with a fluid such as water or a saline solution. Wetting the graft and the first substrate provides lubrication between the graft and the first substrate and allows for easy transfer of the graft from the first substrate to the second substrate. After wetting the first substrate, the graft has a greater affinity for the second substrate than the first substrate. The wetted first substrate is then removed from the second substrate and the grafts remain adhered to the second substrate.

Upon transfer, the stratum corneum layer of the graft becomes adhered to the second substrate, and the basal layer is orientated away from the second substrate. Such a technique ensures that the basal layer of the graft is oriented away from the second substrate and is thus available to be transfected with one or more nucleic acid sequences and to interact with the recipient site of a patient.

Another embodiment of the invention provides a devices for obtaining a skin graft. Devices of the invention include a hollow body having a distal end configured for placement on skin, a mechanism for raising a blister, and a cutter integrated in the body for cutting the blister produced on the skin.

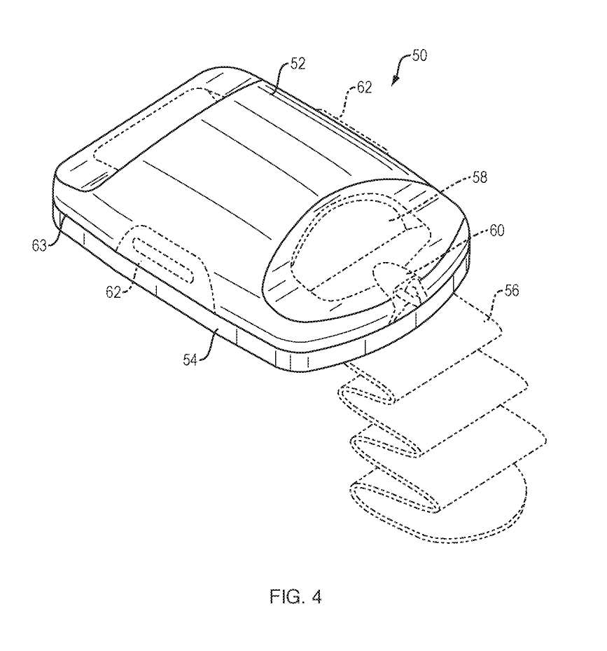

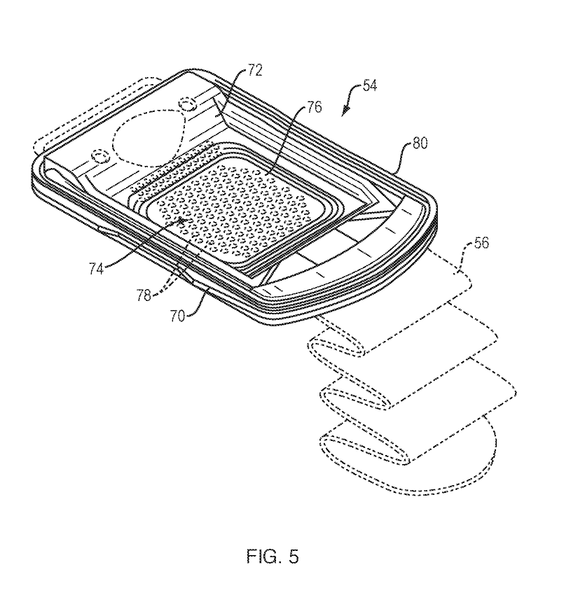

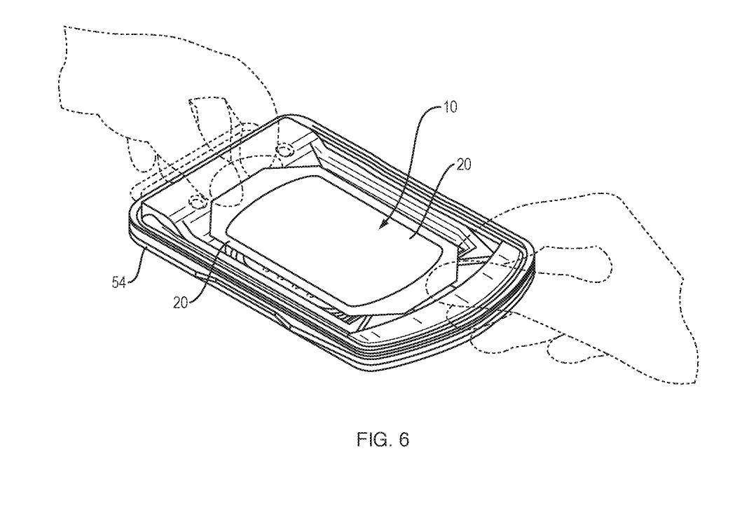

FIG. 4 is a schematic view of a skin graft harvester 50 for use with an absorbent substrate in accordance with various embodiments of the present teachings. In this illustrative embodiment, the harvest 50 includes a detachable head portion 52 and harvester body 54. The harvester body 54 is adapted for placement on a patient's skin at a donor site where skin grafts are to be obtained, e.g., on the inner thigh, and secured in place, for example, with strap 56 (shown in phantom). The head 52 can further include a heater (not shown) powered via a coupler 60 adapted to couple with a power source in a base unit (not shown). The head 52 further includes a seal 63 which permits a reduced pressure chamber to be formed when the head 52 and body 54 are joined together and the harvester 50 is coupled to a vacuum pump or other source of reduced pressure, e.g., via coupler 60 connecting the harvester 50 to its base unit. The head 52 can further include one or more windows 58 for observation of skin blisters being formed within the chamber by application of reduced pressure, heat or both. Once the blisters have been formed, the head 52 can be removed, e.g., by deactivating the source of reduced pressure and by actuation of release levers 62, which break the seal 63 and allow the head 52 to be lifted off the harvester body 54.

FIG. 5 is a schematic view of the skin graft harvester 50 of FIG. 4 with the head 52 removed and the cutting mechanism 74 exposed. The harvester body 54 can include a base portion 70, a sled 72, and actuator handle 80. The cutting mechanism 74 can include a plurality of plates with initially aligned holes through which skin blisters are drawn by heat and/or application of suction when the head 52 is joined to the harvester body 54 and activated. Once the blisters are formed, they can be cleaved by the cutting mechanism 74. For example, below the top plate depicted in FIG. 1, one or more additional plates, e.g., a cutter plate and a bottom plate can be deployed with aligned holes. By actuation (e.g., pulling up) of handle 80, the sled 72 is caused to move horizontally such that one of the plates below the top plate, e.g., the “cutter plate” (not shown) also moves (because of its linkage to the sled 72), thereby occluding the alignment of holes 78 and cleaving the raised blisters from the donor's skin.

FIG. 6 is a schematic view of the skin graft harvester 50 of FIG. 4 with a substrate 10 deployed in the harvester body 54 to capture skin grafts. The substrate can, for example, be a Tegaderm® substrate, an Adaptic Touch™ substrate, or a similar adherent or tacky sheet material. In the illustrated embodiment, the user (e.g., clinician) places the substrate 10 in the harvester holding the backing 22 with the sealing member 20 upwards and the base layer (not visible) in contact with the top plate of cutter mechanism (as shown in FIG. 1). By so placing the substrate, the base layer will also come into contact with the skin blisters. In one preferred embodiment, the substrate is so situated before the cutter mechanism is actuated to cleave the blisters into skin grafts (as described above). In other embodiments, the substrate can be placed onto the harvester after cleavage to capture grafts that have already been cleaved from the skin. In either event the substrate can then be removed from the harvester body 54 and applied to a recipient site, as illustrated in FIG. 4.

In certain embodiments, the present invention relates to a skin graft comprising one or more cells (e.g., stem cells, basal cells, Langerhans cells, Merkel cells, epidermal stem cells, epithelial cells, keratinocytes, epidermal cells, melanocytes, epithelial progenitor cells, etc.). Further, the one or more cells can comprise at least one nucleic acid sequence (e.g., an exogenous sequence). Also, the nucleic acid sequence can modulate a cellular response, for example, enhancing an epithelialization rate. The nucleic acid sequence can also comprise a fluorescent tag sequence or fluorophore. Expression of the fluorescent tag sequence (e.g., a GFP sequence or variant there) can allow the visualization and/or monitoring of re-epithelization and/or repigmentation of the wound site. The skin grafts described herein can be used for the treatment of skin disorders, including cuts, burns, trauma, disease (e.g., epidermal metabolic disorders), and depigmentation.

In certain embodiments, 1, 2, 3, 4, 5, 6, 7, 8, 9, 10 or more nucleic acid sequences are transfected into cells of the skin graft. Each nucleic acid sequence encodes a particular protein, growth factor, hormone, cytokine, etc., which modulates (e.g., stimulates, regulates, activates, etc.) a cellular response. The nucleic acid sequence can also encode a particular protein that can fluoresce, such as, for example, green fluorescent protein and variants of GFP. Other exemplary fluorescent proteins that can be used to monitor and visualize wound healing are also included in Table 1, below.

TABLE 1
List of Fluorescent Proteins

| | | | | | | |
|---|---|---|---|---|---|---|
| | | λₑₘ | | | | |
| Protein | λₑₓ (nm) | (nm) | EC | QY | Brightness | pKa |

UV Proteins

| | | | | | | |
|---|---|---|---|---|---|---|
| Sirius | 355 | 424 | 15000 | 0.24 | 3.6 | 3 |

Blue Proteins

| | | | | | | |
|---|---|---|---|---|---|---|
| Azurite | 383 | 450 | 26200 | 0.55 | 14.4 | 5 |
| EBFP2 | 383 | 448 | 32000 | 0.56 | 18 | 4.5 |
| mKalama1 | 385 | 456 | 36000 | 0.45 | 16 | 5.5 |
| mTagBFP2 | 399 | 454 | 50600 | 0.64 | 32.4 | 2.7 |
| TagBFP | 402 | 457 | 52000 | 0.63 | 32.8 | 2.7 |

Cyan Proteins

| | | | | | | |
|---|---|---|---|---|---|---|
| ECFP | 433 | 475 | 32500 | 0.4 | 13 | 4.7 |
| Cerulean | 433 | 475 | 43000 | 0.62 | 26.7 | 4.7 |
| mCerulean3 | 433 | 475 | 40000 | 0.8 | 32 | 4.7 |
| SCFP3A | 433 | 474 | 30000 | 0.56 | 16.8 | 4.5 |
| CyPet | 435 | 477 | 35000 | 0.51 | 17.8 | 5.02 |
| mTurquoise | 434 | 474 | 30000 | 0.84 | 25.2 | |
| mTurquoise2 | 434 | 474 | 30000 | 0.93 | 27.9 | 3.1 |
| TagCFP | 458 | 480 | 37000 | 0.57 | 21 | 4.7 |
| mTFP1 | 462 | 492 | 64000 | 0.85 | 54 | 4.3 |
| monomeric | 470 | 496 | 22150 | 0.7 | 15.5 | 7 |
| Midoriishi- | | | | | | |
| Cyan | | | | | | |
| Aquamarine | 430 | 474 | 26000 | 0.89 | 23.1 | 3.3 |

Green Proteins

| | | | | | | |
|---|---|---|---|---|---|---|
| TurboGFP | 482 | 502 | 70000 | 0.53 | 37.1 | 5.2 |
| TagGFP2 | 483 | 506 | 56500 | 0.6 | 33.9 | 4.7 |
| mUKG | 483 | 499 | 60000 | 0.72 | 43.2 | 5.2 |
| Superfolder | 485 | 510 | 83300 | 0.65 | 54.1 | |
| GFP | | | | | | |
| Emerald | 487 | 509 | 57500 | 0.68 | 37.3 | 6 |
| EGFP | 488 | 507 | 56000 | 0.6 | 33.6 | 6 |
| Monomeric | 492 | 505 | 55000 | 0.74 | 40.7 | 5.8 |
| Azami Green | | | | | | |
| mWasabi | 493 | 509 | 70000 | 0.8 | 56 | 6 |
| Clover | 505 | 515 | 111000 | 0.76 | 84.4 | 6.1 |
| mNeonGreen | 506 | 517 | 116000 | 0.8 | 92.8 | 5.7 |

Yellow Proteins

| | | | | | | |
|---|---|---|---|---|---|---|
| TagYFP | 508 | 524 | 64000 | 0.62 | 39.7 | 5.5 |
| EYFP | 513 | 527 | 83400 | 0.61 | 50.9 | 6.9 |
| Topaz | 514 | 527 | 94500 | 0.6 | 56.7 | |
| Venus | 515 | 528 | 92200 | 0.57 | 52.5 | 6 |
| SYFP2 | 515 | 527 | 101000 | 0.68 | 68.7 | 6 |
| Citrine | 516 | 529 | 77000 | 0.76 | 58.5 | 5.7 |
| Ypet | 517 | 530 | 104000 | 0.77 | 80.1 | 5.6 |
| lanRFP-ΔS83 | 521 | 592 | 71000 | 0.1 | 7.1 | 4.7 |
| mPapaya1 | 530 | 541 | 43000 | 0.83 | 35.7 | 6.8 |

Orange Proteins

| | | | | | | |
|---|---|---|---|---|---|---|
| Monomeric | 548 | 559 | 51600 | 0.6 | 31 | 5 |
| Kusabira- | | | | | | |
| Orange | | | | | | |
| mOrange | 548 | 562 | 71000 | 0.69 | 49 | 6.5 |
| m0range2 | 549 | 565 | 58000 | 0.6 | 34.8 | 6.5 |
| mKOκ | 551 | 563 | 105000 | 0.61 | 64 | 4.2 |
| mKO2 | 551 | 565 | 63800 | 0.62 | 39.6 | 5.5 |

Red Proteins

| | | | | | | |
|---|---|---|---|---|---|---|
| TagRFP | 555 | 584 | 100000 | 0.48 | 49 | 3.8 |
| TagRFP-T | 555 | 584 | 81000 | 0.41 | 33.2 | 4.6 |
| mRuby | 558 | 605 | 112000 | 0.35 | 39.2 | 4.4 |
| mRuby2 | 559 | 600 | 113000 | 0.38 | 43 | 5.3 |
| mTangerine | 568 | 585 | 38000 | 0.3 | 11.4 | 5.7 |
| mApple | 568 | 592 | 75000 | 0.49 | 36.7 | 6.5 |
| mStrawberry | 574 | 596 | 90000 | 0.29 | 26.1 | 4.5 |
| FusionRed | 580 | 608 | 95000 | 0.19 | 18.1 | 4.6 |
| mCherry | 587 | 610 | 72000 | 0.22 | 15.8 | 4.5 |
| mNectarine | 558 | 578 | 58000 | 0.45 | 26.1 | 6.9 |

Far Red Proteins

| | | | | | | |
|---|---|---|---|---|---|---|
| mKate2 | 588 | 633 | 62500 | 0.4 | 25 | 5.4 |
| HcRed- | 590 | 637 | 160000 | 0.04 | 6.4 | |
| Tandem | | | | | | |
| mPlum | 590 | 649 | 41000 | 0.1 | 4.1 | 4.5 |
| mRaspberry | 598 | 625 | 86000 | 0.15 | 12.9 | |
| mNeptune | 600 | 650 | 67000 | 0.2 | 13.4 | 5.4 |
| NirFP | 605 | 670 | 15700 | 0.06 | 0.9 | 4.5 |
| TagRFP657 | 611 | 657 | 34000 | 0.1 | 3.4 | 5 |
| TagRFP675 | 598 | 675 | 46000 | 0.08 | 3.7 | 5.7 |
| mCardinal | 604 | 659 | 87000 | 0.19 | 16.5 | 5.3 |

Near IR Proteins

| | | | | | | |
|---|---|---|---|---|---|---|
| iFP1.4 | 684 | 708 | 92000 | 0.07 | 6.4 | 4.6 |
| iRFP713 | 690 | 713 | 105000 | 0.06 | 6.2 | 4 |
| (iRFP) | | | | | | |
| iRFP670 | 643 | 670 | 114000 | 0.11 | 12.5 | 4 |
| iRFP682 | 663 | 682 | 90000 | 0.11 | 9.9 | 4.5 |
| iRFP702 | 673 | 702 | 93000 | 0.08 | 7.4 | 4.5 |
| iRFP720 | 702 | 720 | 96000 | 0.06 | 5.8 | 4.5 |
| iFP2.0 | 690 | 711 | 86000 | 0.08 | 6.9 | |
| mIFP | 683 | 704 | 82000 | 0.08 | 6.6 | 3.5 |

Sapphire-type Proteins

| | | | | | | |
|---|---|---|---|---|---|---|
| Sapphire | 399 | 511 | 29000 | 0.64 | 18.6 | |
| T-Sapphire | 399 | 511 | 44000 | 0.6 | 26.4 | 4.9 |
| mAmetrine | 406 | 526 | 45000 | 0.58 | 26.1 | 6 |

Long Stokes Shift Proteins

| | | | | | | |
|---|---|---|---|---|---|---|
| mKeima Red | 440 | 620 | 14400 | 0.24 | 3.5 | 6.5 |
| mBeRFP | 446 | 611 | 65000 | 0.27 | 17.6 | 5.6 |
| LSS-mKate2 | 460 | 605 | 26000 | 0.17 | 4.4 | 2.7 |
| LSS-mKate1 | 463 | 624 | 31200 | 0.08 | 2.5 | 3.2 |
| LSSmOrange | 437 | 572 | 52000 | 0.45 | 23.4 | 5.7 |

For example, the at least one nucleic acid sequence can comprise an activin sequence, an antisense-miRNA sequence, a microRNA family sequence, a β-nerve growth factor sequence, a chemokine sequence, an epidermal growth factor sequence, a fibroblast growth factor sequence, a hepatocyte growth factor sequence, an insulin-like growth factor sequence, an interleukin sequence, a keratinocyte growth factor 1 sequence, a neuregulin sequence, a platelet derived growth factor sequence, a transforming growth factor α sequence, a transforming growth factor β1 or β2 sequence, a vascular endothelial growth factor sequence, a β-3,4-dihydroxyphenylalanine (DOPA) sequence a melanogenesis producing gene (e.g., a monophenol monooxygenase sequence, a 3,4-β-dihydroxyphenylalanine oxygen oxidoreductase sequence, a tyrosinase-related protein 1 (TYRP1) sequence, a DOPAchrome tautomerase (DCT) sequence), an endothelin-1 (ET-1) sequence, a proopiomelanocortin (POMC) sequence, a melanocyte-stimulating hormone (MSH) sequence, a fluorescence protein coding sequence (e.g., green fluorescent protein (GFP) and GPF variants such as, yellow fluorescent protein, cyan fluorescent protein, blue fluorescent protein or red fluorescent protein), or a combination thereof.

The nucleic acid sequence(s) can be an exogenous or endogenous sequence. In some embodiments, the at least one nucleic acid sequence comprises a DNA sequence, a RNA sequence, or a combination thereof. In some embodiments, the nucleic acid is single-stranded, double-stranded or a combination thereof. In some embodiments, wherein the nucleic acid is RNA, the RNA can be any of miRNA, mRNA, tRNA, rRNA, and siRNA. In some embodiments, the nucleic acid is linear, circular (e.g., plasmid, mitochondrial DNA), or a combination thereof.

It will be readily appreciated by one of ordinary skill in the art, that the devices, systems, and methods described herein can be applied to transfection of one or more amino acid sequences. For example, transfection of one or more nucleic acid sequences typically requires expression of that sequence to have a biological effect. In some embodiments, intact functional proteins and peptides and other amino acid sequences can be transfected into one or more cells of a skin graft.

For example, 1, 2, 3, 4, 5, 6, 7, 8, 9, 10 or more amino acid sequences are transfected into cells of the skin graft. Each amino acid sequence is a particular protein, peptide, growth factor, hormone, cytokine, etc., which modulates (e.g., stimulates, regulates, activates, etc.) a cellular response. The amino acid sequence can also be for a particular fluorescent protein, such as those listed in Table 1.

For example, the amino acid sequence can comprise an activin sequence, an antisense-miRNA sequence, a microRNA family sequence, a β-nerve growth factor sequence, a chemokine sequence, an epidermal growth factor sequence, a fibroblast growth factor sequence, a hepatocyte growth factor sequence, an insulin-like growth factor sequence, an interleukin sequence, a keratinocyte growth factor 1 sequence, a neuregulin sequence, a platelet derived growth factor sequence, a transforming growth factor α sequence, a transforming growth factor β1 or β2 sequence, a vascular endothelial growth factor sequence, a β-3,4-dihydroxyphenylalanine (DOPA) sequence a melanogenesis producing gene (e.g., a monophenol monooxygenase sequence, a 3,4-β-dihydroxyphenylalanine oxygen oxidoreductase sequence, a tyrosinase-related protein 1 (TYRP1) sequence, a DOPAchrome tautomerase (DCT) sequence), an endothelin-1 (ET-1) sequence, a proopiomelanocortin (POMC) sequence, a melanocyte-stimulating hormone (MSH) sequence, a fluorescence coding sequence (e.g., green fluorescent protein (GFP) and GFP variants such as, yellow fluorescent protein, cyan fluorescent protein, blue fluorescent protein or red fluorescent protein), or a combination thereof.

In the systems and methods described herein, the at least one nucleic acid sequence can be introduced into one or more cells of a skin graft by a transfection method. Also, the at least one amino acid sequence can be introduced into one or more cells of a skin graft by a transfection method. For example, the transfection method can be a chemical transfection and/or a non-chemical transfection.

For example, a chemical transfection method includes, but is not limited to, calcium phosphate transfection, lipofection (e.g., liposome-mediated transfection, cationic lipid transfection), cationic polymer transfection (e.g., DEAE-dextran mediated transfection) and cationic amino acid transfection.

Non-limiting examples of non-chemical transfection methods are electroporation, sonoporation, laser-mediated (e.g., optical) transfection, direct injection (e.g., microinjection), magnetofection, impalefection, biolistic particle delivery transfection, viral delivery, and receptor-mediated uptake.

As described herein, harvesting one or more skin grafts from a donor site comprises one or more cells from an epidermis. For example, the one or more cells from the epidermis can comprise an epithelial cell (e.g., a stratified squamous epithelial cell), a keratinocyte, a basal cell, a melanocyte, a Langerhans cell, a Merkel cell, a epidermal stem cell, or combinations thereof.

In various embodiments, the skin graft comprises an autologous skin graft, an allograft, or a xenograft.

In some embodiments, the systems and methods described herein relate to transfecting cells to achieve a particular cellular response. Also, cells can be transfected with a fluorescent protein (e.g., a nucleic acid sequence that encodes a fluorescent protein or an amino acid sequence) to allow for visualization (via the fluorescence) of wound healing and/or closure. Particularly, one or more nucleic acid sequences can be introduced into (one or more) skin graft cells, e.g., cells harvested for a skin graft, using a known transfection technique. Those transfected cells can be genetically modified such that expression of the one or more nucleic acid sequences modulates a cellular response. For example, a cellular response can comprise an enhanced epithelialization rate, an enhanced pigmentation rate, or a combination thereof. See Table 2, below, for an exemplary list of agents/factors and known cellular response/function.

TABLE 2
Examples of sequences for transfection

| Biological Agent | Function |
|---|---|
| Activins | Enhance wound repair |
| Anti-sense-miRNA (e.g., miRNA-210) | Down-regulation is beneficial for healing |
| microRNA expressed in skin; mi-RNA-152, | Skin development and epidermal |
| 143, 126, 21, 27a, 214, 16, 125, 34, 205, 27, | differentiation |
| 30, 191, 200, 199, 19 | |
| β-Nerve Growth Factor | Stimulates wound healing |
| Chemokine families (e.g., CXCR1-4 and | Stimulates wound healing |
| CCR1-5) | |
| Epidermal Growth Factor | Promotes epidermal cell proliferation, |
| | migration, differentiation and wound repair |
| Fibroblast Growth Factor (FGF1, FGF2, | Chemotactic and mitogenic for fibroblasts |
| FGF4, FGF7, FGF10) | and keratinocytes, stimulates angiogenesis |
| Insulin-like Growth Factor | |
| Interleukins: IL1, IL-3, IL-6, IL-8, IL10 | Stimulates wound healing |
| family cytokines | |
| Keratinocyte Growth Factor 1 (FGF7) and 2 | Promotes epidermal cell proliferation, |
| (FGF10) | migration, differentiation and wound repair |
| Neuregulins (NDF1-4) | Stimulates wound healing |
| Platelet Derived Growth Factor | |
| Transforming Growth Factor α | Stimulates keratinocyte and fibroblast |
| | migration |
| Transforming Growth Factor β1,2 | Stimulates angiogenesis and cell |
| | proliferation |
| Vascular Endothelial Growth Factor | Stimulates angiogenesis |
| β-3,4-dihydroxyphenylalanine (DOPA) | Stimulate melanogenesis |
| monophenol, 3,4-β-dihydroxyphenylalanine | Stimulate melanogenesis |
| oxygen oxidoreductase | |
| tyrosinase-related protein 1 (TYRP1) | Stimulate melanogenesis |
| DOPAchrome tautomerase (DCT) | Stimulate melanogenesis |
| endothelin-1 (ET-1) | Stimulate melanogenesis |
| proopiomelanocortin (POMC) | Stimulate melanogenesis |
| melanocyte-stimulating hormone (MSH) | Stimulate melanogenesis |
| fluorescence coding sequences (e.g., green | Epidermal cell visualization |
| (GFP), yellow (YFP), red (RFP), blue (BFP), | |
| cyan (CFP) fluorescent protein) | |

### EXEMPLIFICATION

### Example 1

The methods described herein allow for the introduction of one or more factors (e.g., nucleic acids, amino acids, proteins, etc.) that can revitalize autologous epidermis from aged or unhealthy patients to restore effective (re)epithelialization rates and/or (re)pigmentation rates. The methods can also allow for more effective epidermal grafting, faster time to wound closure, decreased recovery time, etc. Autologous epidermis harvested from donor sites of aged patients or from patients with underlying health conditions and/or disease may yield suboptimal grafts in their ability to (re)epithelialize a wound. The methods can also allow for the monitoring of and visualization of wound healing (e.g., repigmentation and/or reepithelialization) using the transfected cells described herein.

Chronic wounds are often observed in elderly patients and/or in patients with severe comorbidities. Impaired (re)epithelialization is a hallmark of these hard to heal wounds. Autologous epidermal grafting provides a solution to wound closure by transferring the patient's own epidermal cells to the recalcitrant wound site. In the geriatric population and in the severely unhealthy patient, autologous epidermal grafts may not have the optimal vitality to (re)epithelialize a wound as effectively as a epidermal graft from a healthy, young individual. Chronically aged skin has been characterized and shown to have diminished expression of growth factors, extracellular matrix components, proliferation and migratory capacity. Analysis of underlying biochemical changes in aged skin have previously demonstrated a decrease in activation of certain vulnerary genes, including transforming growth factor-beta 1, CTGF, and KGF. The methods described herein can also allow for the visualization and/or monitoring of epidermal grafts as they migrate and proliferate to achieve wound closure.

### Protocol 1:

Referring to FIGS. 7 and 8A, epidermal grafts can be formed 910 and harvested 930 while maintaining a fixed orientation as described herein. Before, during or after the skin graft harvesting, transfection reagents at transfection station 920 can be prepared. A donor site can be prepared for blister formation. After placing a skin graft harvesting device on the donor site, microdome formation 910 can be assessed in about 30 minutes. Skin grafts are then harvested and acquired 930. The epidermal grafts are oriented so that the outer stratum corneum layer is adhered to the transfer dressing 940 (e.g., any film, foam, substrate or material with adhesive properties), and stratum basale is exposed (i.e., facing away from the dressing). Also, the harvested skin grafts can be transferred with other types of transfer dressings 940 such as Tegaderm®, Adaptic Touch™, or any non-absorbent dressing to the transfection station reservoir 950.

During blister formation, as illustrated in FIG. 8A, a source of exogenous nucleic acid (e.g., DNA and/or RNA in “Tube 2”) and transfection reagents (e.g., cationic polymer, calcium phosphate, cationic lipid or cationic amino acid in “Tube 1”) in a cell media are mixed (“transfection cocktail”) and placed in a transfection station reservoir. The transfection station reservoir contains all of the necessary reagents to perform a chemical transfection (e.g., transfection cocktail). The transfection station reservoir can be any reservoir configured and sized for the transfection of one or more cells in or derived from a skin graft. The transfection station reservoir can also be temperature and/or pH controlled.

In a chemical transfection (steps 920 and 950 of FIG. 7; see also FIG. 8B), transfection reagents can include, for example, a cationic or amphipathic lipid to form a miscelle, or a liposome (e.g., in a 1:1; 1:2; 1:4; 1:10; 1:100 ratio) in the presence of a polycationic compound (e.g., hexamethrine bromide, poly-L-amino acid salts, poly-D-amino acid salts, and polyelectrolytes; in a concentration of about 1 μg/ml to about 200 μg/ml) in standard cell medium for about 30 minutes to about 4 hours. Suitable cationic liposomes mixtures are commercially available, including, for example: Lipofectin®, and LipofectAMINE® (Thermofisher Scientific, Grand Island, N.Y.).

Referring to FIGS. 7 and 8B, the epidermal grafts are incubated in the transfection station 950 for a sufficient period of time for transfection of the nucleic acid sequence (e.g., 0.1-1 μg/μl) and/or amino acid sequence to occur in one or more cells of the skin grafts, e.g., for about 30 minutes. The transfection time can vary and depend on a number of factors, such as the type of transfection method being used, the type of exogenous nucleic acid (e.g., DNA or RNA), in the size of exogenous nucleic acid (e.g., length number of bases), thickness of the skin graft, and others.

### Protocol 2:

Alternatively, step 920 of FIG. 7, can include placing the harvested epidermal grafts in a transfection station reservoir 950 that is embedded with electrodes (e.g., gold electrodes) for electroporation (i.e., non-chemical transfection). For example, the transfection station is configured to hold a conductive solution (e.g., cell media or buffer) containing at least one exogenous nucleic acid sequence (e.g., a DNA or a RNA sequence) and/or amino acid sequence (e.g., peptide, protein). A reservoir lid is embedded with electrodes (e.g., gold electrodes) and can be connected to the transfection station base electrodes. The transfection station is adapted to be connected to a programmable power source (e.g., DC power). For example, the reservoir lid can be connected to a programmable power source, which can provide a range of pulse frequencies. For instance, a range of pulse frequencies can be used to electroporate the epidermal grafts for certain lengths of time. An electrical pulse at an optimized voltage can be discharged through the transfection station. This induces temporary pores in the membranes of the skin graft cells for nucleic acid (and/or amino acid) entry. The pulse frequencies can be used to electroporate the skin grafts for a certain period of time.

The transfection station reservoir can be placed in an insulator chamber for all or part of the electroporation procedure to maintain the reservoir at a certain temperature. This can minimize damage to the skin graft cells during the electroporation protocol. Once cells are transfected with the at least one exogenous nucleic acid sequence, the transfected cells can be allowed to recover.

Before, during, or after a chemical or a non-chemical transfection, a recipient site can be prepared for receiving the transfected skin graft. After any suitable transfection technique, such as those described in Protocol 1 and Protocol 2, the transfected epidermal cells can be grafted on to a prepared recipient site.

Referring to FIG. 9, a harvested skin graft 200 attached to a substrate 100 and a fluorescent protein sequence 400 (e.g., a Green Fluorescent Protein construct or a GFP variant) can be added to the transfection station reservoir 300 as described and illustrated in FIGS. 7 and 8A. The transfection station 300 can also have one or more additional exogenous nucleic acid sequences in addition to the fluorescent protein sequence 400. The GFP sequence 400 or GFP variant sequence is introduced (transfected) into one or more cells 211 of the basal layer in the epidermal graft 200 by any one of the transfection methods described herein, such as chemical transfection or non-chemical transfection.

The transfected skin graft cells 211 can then be used to treat a skin wound, such as a burn or cut. After grafting the GFP-transfected epidermal skin graft, wound healing (e.g., re-epithelialization, repigmenation, etc.) can be monitored by visualizing cells 211 that have been transfected with and express GFP. This allows for the monitoring of skin wound closure. The types of fluorescent tags or markers used in the methods and systems described herein can include, for example, GFP and variants of GFP, such as YFP, BFP and CFP, or any fluorescent protein such as those listed in Table 1. Other known methods of tagging or labeling one or more cells are known and appreciated to those of ordinary skill in the art. For example, chemical reagents, such as chromophores can be used to tag, label or visualize one or more cells in a skin graft.

### Example 2

Activin A Homo sapiens coding sequence (SEQ ID NO:1):

ATGGTAGATGGAGTGATGATTCTTCCTGTGCTTATCATGATTGCTCTCCC
CTCCCCTAGTATGGAAGATGAGAAGCCCAAGGTCAACCCCAAACTCTACA
TGTGTGTGTGTGAAGGTCTCTCCTGCGGTAATGAGGACCACTGTGAAGGC
CAGCAGTGCTTTTCCTCACTGAGCATCAACGATGGCTTCCACGTCTACCA
GAAAGGCTGCTTCCAGGTTTATGAGCAGGGAAAGATGACCTGTAAGACCC
CGCCGTCCCCTGGCCAAGCCGTGGAGTGCTGCCAAGGGGACTGGTGTAAC
AGGAACATCACGGCCCAGCTGCCCACTAAAGGAAAATCCTTCCCTGGAAC
ACAGAATTTCCACTTGGAGGTTGGCCTCATTATTCTCTCTGTAGTGTTCG
CAGTATGTCTTTTAGCCTGCCTGCTGGGAGTTGCTCTCCGAAAATTTAAA
AGGCGCAACCAAGAACGCCTCAATCCCCGAGACGTGGAGTATGGCACTAT
CGAAGGGCTCATCACCACCAATGTTGGAGACAGCACTTTAGCAGATTTAT
TGGATCATTCGTGTACATCAGGAAGTGGCTCTGGTCTTCCTTTTCTGGTA
CAAAGAACAGTGGCTCGCCAGATTACACTGTTGGAGTGTGTCGGGAAAGG
CAGGTATGGTGAGGTGTGGAGGGGCAGCTGGCAAGGGGAGAATGTTGCCG
TGAAGATCTTCTCCTCCCGTGATGAGAAGTCATGGTTCAGGGAAACGGAA
TTGTACAACACTGTGATGCTGAGGCATGAAAATATCTTAGGTTTCATTGC
TTCAGACATGACATCAAGACACTCCAGTACCCAGCTGTGGTTAATTACAC
ATTATCATGAAATGGGATCGTTGTACGACTATCTTCAGCTTACTACTCTG
GATACAGTTAGCTGCCTTCGAATAGTGCTGTCCATAGCTAGTGGTCTTGC
ACATTTGCACATAGAGATATTTGGGACCCAAGGGAAACCAGCCATTGCCC
ATCGAGATTTAAAGAGCAAAAATATTCTGGTTAAGAAGAATGGACAGTGT
TGCATAGCAGATTTGGGCCTGGCAGTCATGCATTCCCAGAGCACCAATCA
GCTTGATGTGGGGAACAATCCCCGTGTGGGCACCAAGCGCTACATGGCCC
CCGAAGTTCTAGATGAAACCATCCAGGTGGATTGTTTCGATTCTTATAAA
AGGGTCGATATTTGGGCCTTTGGACTTGTTTTGTGGGAAGTGGCCAGGCG
GATGGTGAGCAATGGTATAGTGGAGGATTACAAGCCACCGTTCTACGATG
TGGTTCCCAATGACCCAAGTTTTGAAGATATGAGGAAGGTAGTCTGTGTG
GATCAACAAAGGCCAAACATACCCAACAGATGGTTCTCAGACCCGACATT
AACCTCTCTGGCCAAGCTAATGAAAGAATGCTGGTATCAAAATCCATCCG
CAAGACTCACAGCACTGCGTATCAAAAAGACTTTGACCAAAATTGATAAT
TCCCTCGACAAATTGAAAACTGACTGTTGA

Epidermal Growth Factor Homo sapiens coding sequence (SEQ ID NO:2):

ATGCTGCTCACTCTTATCATTCTGTTGCCAGTAGTTTCAAAATTTAGTTT
TGTTAGTCTCTCAGCACCGCAGCACTGGAGCTGTCCTGAAGGTACTCTCG
CAGGAAATGGGAATTCTACTTGTGTGGGTCCTGCACCCTTCTTAATTTTC
TCCCATGGAAATAGTATCTTTAGGATTGACACAGAAGGAACCAATTATGA
GCAATTGGTGGTGGATGCTGGTGTCTCAGTGATCATGGATTTTCATTATA
ATGAGAAAAGAATCTATTGGGTGGATTTAGAAAGACAACTTTTGCAAAGA
GTTTTTCTGAATGGGTCAAGGCAAGAGAGAGTATGTAATATAGAGAAAAA
TGTTTCTGGAATGGCAATAAATTGGATAAATGAAGAAGTTATTTGGTCAA
ATCAACAGGAAGGAATCATTACAGTAACAGATATGAAAGGAAATAATTCC
CACATTCTTTTAAGTGCTTTAAAATATCCTGCAAATGTAGCAGTTGATCC
AGTAGAAAGGTTTATATTTTGGTCTTCAGAGGTGGCTGGAAGCCTTTATA
GAGCAGATCTCGATGGTGTGGGAGTGAAGGCTCTGTTGGAGACATCAGAG
AAAATAACAGCTGTGTCATTGGATGTGCTTGATAAGCGGCTGTTTTGGAT
TCAGTACAACAGAGAAGGAAGCAATTCTCTTATTTGCTCCTGTGATTATG
ATGGAGGTTCTGTCCACATTAGTAAACATCCAACACAGCATAATTTGTTT
GCAATGTCCCTTTTTGGTGACCGTATCTTCTATTCAACATGGAAAATGAA
GACAATTTGGATAGCCAACAAACACACTGGAAAGGACATGGTTAGAATTA
ACCTCCATTCATCATTTGTACCACTTGGTGAACTGAAAGTAGTGCATCCA
CTTGCACAACCCAAGGCAGAAGATGACACTTGGGAGCCTGAGCAGAAACT
TTGCAAATTGAGGAAAGGAAACTGCAGCAGCACTGTGTGTGGGCAAGACC
TCCAGTCACACTTGTGCATGTGTGCAGAGGGATACGCCCTAAGTCGAGAC
CGGAAGTACTGTGAAGATGTTAATGAATGTGCTTTTTGGAATCATGGCTG
TACTCTTGGGTGTAAAAACACCCCTGGATCCTATTACTGCACGTGCCCTG
TAGGATTTGTTCTGCTTCCTGATGGGAAACGATGTCATCAACTTGTTTCC
TGTCCACGCAATGTGTCTGAATGCAGCCATGACTGTGTTCTGACATCAGA
AGGTCCCTTATGTTTCTGTCCTGAAGGCTCAGTGCTTGAGAGAGATGGGA
AAACATGTAGCGGTTGTTCCTCACCCGATAATGGTGGATGTAGCCAGCTC
TGCGTTCCTCTTAGCCCAGTATCCTGGGAATGTGATTGCTTTCCTGGGTA
TGACCTACAACTGGATGAAAAAAGCTGTGCAGCTTCAGGACCACAACCAT
TTTTGCTGTTTGCCAATTCTCAAGATATTCGACACATGCATTTTGATGGA
ACAGACTATGGAACTCTGCTCAGCCAGCAGATGGGAATGGTTTATGCCCT
AGATCATGACCCTGTGGAAAATAAGATATACTTTGCCCATACAGCCCTGA
AGTGGATAGAGAGAGCTAATATGGATGGTTCCCAGCGAGAAAGGCTTATT
GAGGAAGGAGTAGATGTGCCAGAAGGTCTTGCTGTGGACTGGATTGGCCG
TAGATTCTATTGGACAGACAGAGGGAAATCTCTGATTGGAAGGAGTGATT
TAAATGGGAAACGTTCCAAAATAATCACTAAGGAGAACATCTCTCAACCA
CGAGGAATTGCTGTTCATCCAATGGCCAAGAGATTATTCTGGACTGATAC
AGGGATTAATCCACGAATTGAAAGTTCTTCCCTCCAAGGCCTTGGCCGTC
TGGTTATAGCCAGCTCTGATCTAATCTGGCCCAGTGGAATAACGATTGAC
TTCTTAACTGACAAGTTGTACTGGTGCGATGCCAAGCAGTCTGTGATTGA
AATGGCCAATCTGGATGGTTCAAAACGCCGAAGACTTACCCAGAATGATG
TAGGTCACCCATTTGCTGTAGCAGTGTTTGAGGATTATGTGTGGTTCTCA
GATTGGGCTATGCCATCAGTAATGAGAGTAAACAAGAGGACTGGCAAAGA
TAGAGTACGTCTCCAAGGCAGCATGCTGAAGCCCTCATCACTGGTTGTGG
TTCATCCATTGGCAAAACCAGGAGCAGATCCCTGCTTATATCAAAACGGA
GGCTGTGAACATATTTGCAAAAAGAGGCTTGGAACTGCTTGGTGTTCGTG
TCGTGAAGGTTTTATGAAAGCCTCAGATGGGAAAACGTGTCTGGCTCTGG
ATGGTCATCAGCTGTTGGCAGGTGGTGAAGTTGATCTAAAGAACCAAGTA
ACACCATTGGACATCTTGTCCAAGACTAGAGTGTCAGAAGATAACATTAC
AGAATCTCAACACATGCTAGTGGCTGAAATCATGGTGTCAGATCAAGATG
ACTGTGCTCCTGTGGGATGCAGCATGTATGCTCGGTGTATTTCAGAGGGA
GAGGATGCCACATGTCAGTGTTTGAAAGGATTTGCTGGGGATGGAAAACT
ATGTTCTGATATAGATGAATGTGAGATGGGTGTCCCAGTGTGCCCCCCTG
CCTCCTCCAAGTGCATCAACACCGAAGGTGGTTATGTCTGCCGGTGCTCA
GAAGGCTACCAAGGAGATGGGATTCACTGTCTTGACTCTACTCCACCCCC
TCACCTCAGGGAAGATGACCACCACTATTCCGTAAGAAATAGTGACTCTG
AATGTCCCCTGTCCCACGATGGGTACTGCCTCCATGATGGTGTGTGCATG
TATATTGAAGCATTGGACAAGTATGCATGCAACTGTGTTGTTGGCTACAT
CGGGGAGCGATGTCAGTACCGAGACCTGAAGTGGTGGGAACTGCGCCACG
CTGGCCACGGGCAGCAGCAGAAGGTCATCGTGGTGGCTGTCTGCGTGGTG
GTGCTTGTCATGCTGCTCCTCCTGAGCCTGTGGGGGGCCCACTACTACAG
GACTCAGAAGCTGCTATCGAAAAACCCAAAGAATCCTTATGAGGAGTCGA
GCAGAGATGTGAGGAGTCGCAGGCCTGCTGACACTGAGGATGGGATGTCC
TCTTGCCCTCAACCTTGGTTTGTGGTTATAAAAGAACACCAAGACCTCAA
GAATGGGGGTCAACCAGTGGCTGGTGAGGATGGCCAGGCAGCAGATGGGT
CAATGCAACCAACTTCATGGAGGCAGGAGCCCCAGTTATGTGGAATGGGC
ACAGAGCAAGGCTGCTGGATTCCAGTATCCAGTGATAAGGGCTCCTGTCC
CCAGGTAATGGAGCGAAGCTTTCATATGCCCTCCTATGGGACACAGACCC
TTGAAGGGGGTGTCGAGAAGCCCCATTCTCTCCTATCAGCTAACCCATTA
TGGCAACAAAGGGCCCTGGACCCACCACACCAAATGGAGCTGACTCAGTG
A

mir-210 MI0000286 Homo sapiens stem-loop sequence (SEQ ID NO:3):

ACCCGGCAGUGCCUCCAGGCGCAGGGCAGCCCCUGCCCACCGCACACUGC
GCUGCCCCAGACCCACUGUGCGUGUGACAGCGGCUGAUCUGUGCCUGGGC
AGCGCGACCC

mir-152 MI0000286 Homo sapiens stem-loop sequence (SEQ ID NO:4):

UCAGUGCAUGACAGAACUUGG

cloning vector with GFP (SEQ ID NO:5)

TAGTTATTAC TAGCGCTACC GGACTCAGAT CTCGAGCTCA
AGCTTCGAAT TCTGCAGTCG ACGGTACCGC GGGCCCGGGA
TCCACCGGTC GCCACCATGG TGAGCAAGGG CGAGGAGCTG
TTCACCGGGG TGGTGCCCAT CCTGGTCGAG CTGGACGGCG
ACGTAAACGG CCACAAGTTC AGCGTGTCCG GCGAGGGCGA
GGGCGATGCC ACCTACGGCA AGCTGACCCT GAAGTTCATC
TGCACCACCG GCAAGCTGCC CGTGCCCTGG CCCACCCTCG
TGACCACCCT GACCTACGGC GTGCAGTGCT TCAGCCGCTA
CCCCGACCAC ATGAAGCAGC ACGACTTCTT CAAGTCCGCC
ATGCCCGAAG GCTACGTCCA GGAGCGCACC ATCTTCTTCA
AGGACGACGG CAACTACAAG ACCCGCGCCG AGGTGAAGTT
CGAGGGCGAC ACCCTGGTGA ACCGCATCGA GCTGAAGGGC
ATCGACTTCA AGGAGGACGG CAACATCCTG GGGCACAAGC
TGGAGTACAA CTACAACAGC CACAACGTCT ATATCATGGC
CGACAAGCAG AAGAACGGCA TCAAGGTGAA CTTCAAGATC
CGCCACAACA TCGAGGACGG CAGCGTGCAG CTCGCCGACC
ACTACCAGCA GAACACCCCC ATCGGCGACG GCCCCGTGCT
GCTGCCCGAC AACCACTACC TGAGCACCCA GTCCGCCCTG
AGCAAAGACC CCAACGAGAA GCGCGATCAC ATGGTCCTGC
TGGAGTTCGT GACCGCCGCC GGGATCACTC TCGGCATGGA
CGAGCTGTAC AAGTAAAGCG GCCGCGACTC TAGATCATAA
TCAGCCATAC CACATTTGTA GAGGTTTTAC TTGCTTTAAA
AAACCTCCCA CACCTCCCCC TGAACCTGAA ACATAAAATG
AATGCAATTG TTGTTGTTAA CTTGTTTATT GCAGCTTATA
ATGGTTACAA ATAAAGCAAT AGCATCACAA ATTTCACAAA
TAAAGCATTT TTTTCACTGC ATTCTAGTTG TGGTTTGTCC
AAACTCATCA ATGTATCTTA AGGCGTAAAT TGTAAGCGTT
AATATTTTGT TAAAATTCGC GTTAAATTTT TGTTAAATCA
GCTCATTTTT TAACCAATAG GCCGAAATCG GCAAAATCCC
TTATAAATCA AAAGAATAGA CCGAGATAGG GTTGAGTGTT
GTTCCAGTTT GGAACAAGAG TCCACTATTA AAGAACGTGG
ACTCCAACGT CAAAGGGCGA AAAACCGTCT ATCAGGGCGA
TGGCCCACTA CGTGAACCAT CACCCTAATC AAGTTTTTTG
GGGTCGAGGT GCCGTAAAGC ACTAAATCGG AACCCTAAAG
GGAGCCCCCG ATTTAGAGCT

The teachings of all patents, published applications and references cited herein are incorporated by reference in their entirety.

While the preferred embodiments of the invention have been illustrated and described, it will be clear that the invention is not so limited. Numerous modifications, changes, variations, substitutions, and equivalents will occur to those skilled in the art without departing from the spirit and scope of the present invention as defined by the appended claims.

1. A skin graft comprising:
an outer stratum corneum layer; and
an inner basal layer comprising one or more cells, wherein at least one of the one or more cells comprises at least one exogenous nucleic acid sequence, and wherein the at least one exogenous nucleic acid sequence modulates a cellular response that promotes an enhanced epithelialization rate.

2. The skin graft of claim 1, wherein the at least one exogenous nucleic acid sequence comprises a DNA sequence, a RNA sequence, or a combination thereof.

3. The skin graft of claim 1, wherein the skin graft comprises an autologous skin graft.

4. The skin graft of claim 1, wherein the at least one nucleic acid sequence comprises an activin sequence, an antisense-miRNA sequence, a microRNA family sequence, a β-nerve growth factor sequence, a chemokine sequence, an epidermal growth factor sequence, a fibroblast growth factor sequence, a hepatocyte growth factor sequence, an insulin-like growth factor sequence, an interleukin sequence, a keratinocyte growth factor 1 sequence, a neuregulin sequence, a platelet derived growth factor sequence, a transforming growth factor α sequence, a transforming growth factor β1 sequence, a transforming growth factor β2 sequence, a vascular endothelial growth factor sequence, a β-3,4-dihydroxyphenylalanine (DOPA) sequence, a monophenol monooxygenase sequence, a 3,4-β-dihydroxyphenylalanine oxygen oxidoreductase sequence, a tyrosinase-related protein 1 (TYRP1) sequence, a DOPAchrome tautomerase (DCT) sequence, an endothelin-1 (ET-1) sequence, a proopiomelanocortin (POMC) sequence, a melanocyte-stimulating hormone (MSH) sequence, a fluorescence coding sequence, or a combination thereof.

5. The skin graft of claim 4, wherein the fluorescence coding sequence comprises a green fluorescence protein sequence or a variant thereof.

6. The skin graft of claim 1, wherein the at least one exogenous nucleic acid sequence is introduced into one or more cells by a transfection method.

7. The skin graft of claim 6, wherein the transfection method is selected from the group consisting of chemical transfection and non-chemical transfection.

8. The skin graft of claim 7, wherein chemical transfection is selected from the group consisting of a) calcium phosphate transfection, b) lipofection, including liposome-mediated transfection, cationic lipid transfection, c) cationic polymer transfection, including DEAE-dextran mediated transfection, and d) cationic amino acid transfection.

9. The skin graft of claim 7, wherein non-chemical transfection is selected from the group consisting of electroporation, sonoporation, laser-mediated or optical laser-mediated transfection, direct injection, microinjection, magnetofection, impalefection, biolistic particle delivery transfection, viral delivery, and receptor-mediated uptake.

10. The skin graft of claim 2, wherein the one or more cells comprises an epithelial cell, a keratinocyte, a basal cell, a melanocyte, a Langerhans cell, a Merkel cell, an epidermal stem cell, an epithelial progenitor cell, or a combination thereof.

11. A method of treating a skin wound, the method comprising:
harvesting a skin graft, the skin graft comprising one or more cells from a donor site;
introducing at least one nucleic acid sequence into at least one of the one or more cells of said skin graft to produce a transfected skin graft, the nucleic acid sequence modulating a cellular response that promotes an enhanced epithelialization rate, an enhanced repigmentation, or a combination thereof; and
grafting said transfected skin graft to a skin wound to treat the skin wound.

12. The method of claim 11, further comprising transferring the harvested skin graft onto a first substrate.

13. The method of claim 12, wherein a basal layer of the skin graft is oriented so that it is not in contact with the first substrate.

14. The method of claim 12, further comprising transferring the skin graft from the first substrate to a second substrate.

15-26. (canceled)

27. The method of claim 11, wherein the one or more cells is from a basal layer.

28. The method of claim 27, wherein the basal layer comprises an epithelial cell, a keratinocyte, a basal cell, an epithelial progenitor cell, a melanocyte, a Langerhans cell, a Merkel cell, an epidermal stem cell, or a combination thereof.

29. The method of claim 11, further comprising enhancing wound closure after grafting said transfected skin graft to the skin wound on the individual.

30. The method of claim 11, further comprising monitoring a re-epithelialization rate and/or a repigmentation rate of the transfected skin graft.

31-56. (canceled)
